(19) United States
(12) Patent Application Publication
(10) Pub. No.: US 2013/0158028 A1
(43) Pub. Date: Jun. 20, 2013

(54) USE OF STIMULATORS AND ACTIVATORS OF SOLUBLE GUANYLATE CYCLASE FOR TREATING SICKLE-CELL ANEMIA AND CONSERVING BLOOD SUBSTITUTES

(71) Applicants: Johannes-Peter Stasch, Solingen (DE); Eva-Maria Becker, Wuppertal (DE); Hubert Trübel, Wuppertal (DE); Herbert Himmel, Essen (DE)

(72) Inventors: Johannes-Peter Stasch, Solingen (DE); Eva-Maria Becker, Wuppertal (DE); Hubert Trübel, Wuppertal (DE); Herbert Himmel, Essen (DE)

(73) Assignee: BAYER INTELLECTUAL PROPERTY GMBH, Monheim (DE)

(21) Appl. No.: 13/806,270

(22) PCT Filed: Jun. 21, 2011
(86) PCT No.: PCT/EP2011/060331

§ 371 Date: Feb. 28, 2013

(30) Foreign Application Priority Data
Jun. 25, 2010 (EP) 10006623.2

(51) Int. Cl. A61K 31/506; A61K 31/53; A61K 31/426; A61K 31/4439; A61K 31/427; A61K 31/197; A61K 31/5377; A61K 31/519

(52) U.S. Cl. CPC A61K 31/506; A61K 31/5377; A61K 31/53; A61K 31/519; A61K 31/4439; A61K 31/427; A61K 31/197; A61K 31/426
USPC 514/234.2; 514/231.5; 514/236.8; 514/243; 514/248; 514/262.1; 514/265.1; 514/275; 514/341; 514/365; 514/567

(57) ABSTRACT

The present invention relates to the novel use of stimulators and activators of soluble guanylate cyclase for the treatment of sickle cell anemia and for the preservation of blood substitutes. The present invention furthermore relates to the use of stimulators and activators of soluble guanylate cyclase in combination with PDE5 inhibitors for the treatment of sickle cell anemia and for the preservation of blood substitutes.

The present invention relates to the novel use of stimulators and/or activators of soluble guanylate cyclase for the treatment of sickle cell anemia and for the preservation of blood substitutes. The present invention furthermore relates to the use of stimulators and/or activators of soluble guanylate cyclase in combination with PDE5 inhibitors for the treatment of sickle cell anemia and for the preservation of blood substitutes.

In the last decades, the knowledge about the role of NO in blood flow regulation and cardiovascular homeostasis has increased Pulmonary hypertension (PH) is a frequent complication in sickle cell anemia, which is based on a disturbed NO signaling cascade in combination with increased oxidative stress. Here, PH is a complication of sickle cell anemia and a strong predictor of mortality. In sickle cell patients, there is resistance to NO, caused firstly by the immediate scavaging of any NO formed by free hemoglobin (Hb), which is released on degradation of erythrocytes, and secondly also by increased oxidation and degradation of soluble guanylate cyclase (sGC), which is activated by NO as endogenous ligand. For the elucidation of the further pathophysiology, transgenic mice (exclusive expression of human sickle cell hemoglobin) showing various disease symptoms (among others PH) may be of use in particular (Blood. 2007; 109: 3088-3098).

Here, we claim the use of sGC activators and stimulators for treating sickle cell anemia and associated PH, and also other disease symptoms (for example end organ damage affecting brain, kidney or heart).

In addition, the same above-mentioned pathophysiological mechanisms are effective when blood transfusions (for example by storage etc. with an elevated concentration of free Hb) are administered to patients having a transfusion indication.

Furthermore, in the future the combination of an sGC activator and stimulator with a synthetic Hb-based oxygen carrier may mitigate the side effects hitherto observed [Weiskopf, Anaesthesia & Analgesia, 110:3; 659-661, 2010] which are caused by reduced availability of NO, thus allowing clinical application.

sGC activators and stimulators stimulate soluble guanylate cyclase directly, thus replacing the missing effect of NO. sGC stimulators are not affected by free Hb.

Using sGC activators, it is possible to stimulate even oxidized forms of soluble guanylate cyclase directly, independently of NO. This oxidized form may accumulate in relatively high concentrations in tissue exposed to oxidative stress, so that, by using sGC activators, there should also be a targeted treatment of tissue under oxidative stress.

For these reasons, sGC stimulators and sGC activators employed in acute and in particular in chronic situations ought to have positive effects on blood pressure, oxygen saturation and the resulting plasma and tissue cGMP concentrations under experimental conditions. These experimental conditions consist firstly of healthy animals, animals that are being administered external free Hb or else genetically modified animals such as, for example, sickle cell mice (transgenic mice expressing exclusively human sickle cell hemoglobin beta). Here, the experiments are carried out using sGC stimulators and activators “head to head” against the PDE5 inhibitor sildenafil, since sildenafil has recently failed in clinical trials (phase II) in patients suffering from sickle cell anemia. It lead to an increase in pain attacks in the patients, so that the study had to be terminated prematurely. Accordingly, it is imperative to answer the question of whether this is caused by the cGMP increase (in the case of sGC stimulators and activators via stimulation of sGC, in the case of sildenafil via inhibition of the degradation of the cGMP formed by inhibition of phosphodiesterase 5), or whether this is a property of sildenafil or of PDE5 inhibitors in general. The previous clinical trials involving sGC stimulators and activators have hitherto not given any indications of an increased incidence of episodes of pain in the volunteers treated. In contrast, in connection with PDE5 inhibitors such as sildenafil, vardenafil and tadalafil, there have been repeated reports of an increased incidence of back pain. As a consequence, it may be of great interest to be able to differentiate the above-mentioned sGC stimulators and activators from PDE5 inhibitors even at the preclinical phase.

Based on this prior art, it is an object of the present invention to provide substances for treating sickle cell anemia and preserving blood substitutes. This object is achieved by providing the sGC stimulators and/or sGC activators mentioned below alone or in combination and the combination of sGC stimulators and/or sGC activators with PDE5 inhibitors.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyze the biosynthesis of cGMP from guanosine triphosphate (GTP). The known representatives to date of this family can be classified both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. It is of central importance for the activation mechanism. NO is able to bind to the iron atom of heme and thus markedly increase the activity of the enzyme. Heme-free preparations cannot, by contrast, be stimulated by NO. Carbon monoxide (CO) is also able to attach to the central iron atom of heme, but the stimulation by CO is distinctly less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disturbance of the above-mentioned processes. Under pathophysiological conditions, the NO/cGMP system may be suppressed, which may lead for example to high blood pressure, platelet activation, increased cellular proliferation, endothelial dysfunction, atherosclerosis, angina pectoris, heart failure, myocardial infarction, thromboses, stroke and sexual dysfunction.

Owing to the expected high efficiency and few side effects, a treatment of such disorders which targets the influence of the cGMP signal path in organisms and is NO-independent is a promising approach.

Hitherto, for the therapeutic stimulation of the soluble guanylate cyclase use has exclusively been made of compounds such as organic nitrates whose effect is based on NO. This is formed by bioconversion and activates soluble guanylate cyclase by attack at the central iron atom of heme. In addition to the side effects, the development of tolerance is one of the decisive disadvantages of this type of treatment.

In recent years, some substances have been described which stimulate soluble guanylate cyclase directly, i.e. without prior release of NO, such as, for example, 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223), which are to be understood here as sGC stimulators for the purpose of the present invention.

The present invention provides for the use of stimulators and/or activators of soluble guanylate cyclase for the treatment of sickle cell anemia and for the preservation of blood substitutes.

Particular preference is given to the compounds (1)-(26), as shown below:
2-[1-(2-fluorobenzyl)-1H-pyrazolo[3,4-b]pyridin-3-yl]-5-(4-morpholinyl)-4,6-pyrimidinediamine (1), disclosed as example 16 in WO 00/06569,
2-[1-(2-fluorobenzyl)-1H-pyrazolo[3,4-b]pyridin-3-yl]-5-(4-pyridinyl)-4-pyrimidineamine (2), disclosed as example 1 in WO 02/42301,
methyl 4,6-diamino-2-[1-(2-fluorobenzyl)-1H-pyrazolo[3,4-b]pyridin-3-yl]-5-pyrimidinyl(methyl)carbamate (3), disclosed as example 8 in WO 03/095451,
methyl 4,6-diamino-2-[1-(2-fluorobenzyl)-1H-pyrazolo[3,4-b]pyridin-3-yl]-5-pyrimidinylcarbamate (4), disclosed as example 5 in WO 03/095451,
4-({(4-carboxybutyl)[2-(2-{[4-(2-phenylethyl)benzyl]oxy}phenyl)ethyl]amino}methyl)carboxylic acid (5), disclosed as example 8a in WO 01/019780,
methyl {4,6-diamino-2-[5-fluoro-1-(2-fluorobenzyl)-1H-pyrazolo[3,4-b]pyridin-3-yl]pyrimidin-5-yl}carbamate (6),
methyl {4,6-diamino-2-[5-fluoro-1-(2-fluorobenzyl)-1H-pyrazolo[3,4-b]pyridin-3-yl]pyrimidin-5-yl}methylcarbamate (7),
methyl {4,6-diamino-2-[5-fluoro-1-(2-fluorobenzyl)-1H-pyrazolo[3,4-b]pyridin-3-yl]pyrimidin-5-yl}(2,2,2-trifluoroethyl)carbamate (8),
5-chloro-2-5-chlorothiophen-2-sulfonylamino-N-(4-(morpholin-4-sulfonyl)phenyl)benzamide as sodium salt (9), disclosed in WO00/02851,
2-(4-chlorophenylsulfonylamino)-4,5-dimethoxy-N-(4-(thiomorpholin-4-sulfonyl)phenyl)benzamide (10), disclosed in WO00/02851,
1-{6-[5-chloro-2-({4-trans-4-}trifluoromethyl)cyclohexyl]benzyl}oxy)phenyl]pyridin-2-yl}-5-(trifluoromethyl)-1H-pyrazole-4-carboxylic acid (11), disclosed in WO 2009/032249,
1-[6-(2-(2-methyl-4-(4-trifluoromethoxyphenyl)benzyloxy)phenyl)pyridin-2-yl]-5-trifluoromethylpyrazole-4-carboxylic acid (12), disclosed in WO 2009/071504,
1[6-(3,4-dichlorophenyl)-2-pyridinyl-5-(trifluoromethyl)-1H-pyrazole-4-carboxylic acid (13), disclosed in WO 2009/068652,
1-({2-[3-chloro-5-(trifluoromethyl)phenyl]-5-methyl-1,3-thiazol-4-yl}methyl)-1H-pyrazole-4-carboxylic acid (14), 4-({2-[3-(trifluoromethyl)phenyl]-1,3-thiazol-4-yl}methyl)benzoic acid (15) and 1-({2-[2-fluoro-3-(trifluoromethyl)phenyl]-5-methyl-1,3-thiazol-4-yl}methyl)-1H-pyrazole-4-carboxylic acid (16) disclosed in WO 2009/123316,
4-amino-2-[5-chloro-3(3,3,3-trifluoropropyl)-1H-indazol-1-yl]-5,5-dimethyl-5,7-dihydro-6H-pyrrolo[2,3-d]pyrimidin-6-one (17), 4-amino-2[5-chloro-3-(2,3,6-trifluorobenzyl)-1H-indazol-1-yl]-5,5-dimethyl-5,7-dihydro-6H-pyrrolo[2,3-d]pyrimidin-6-one (18), 4-amino-5,5-dimethyl-2-[3-(2,3,6-trifluorobenzyl)1H-thieno[3,4-c]pyrazol-1-yl]-5,7-dihydro-6H-pyrrolo[2,3-d]pyrimidin-6-one (19), 4-amino-5,5-dimethyl-2-[3-(2,3,6-trifluorobenzyl)1H-thieno[2,3-d]pyrazol-1-yl]-5,5-dimethyl-5,7-dihydro-6H-pyrrolo[2,3-d]pyrimidin-6-one (20), 4-amino-5,5-dimethyl-2-[7-(2,3,6-trifluorobenzyl)imidazo[1,5-b]pyridazin-5-yl]-5,7-dihydro-6H-pyrrolo[2,3-d]pyrimidin-6-one (21), 4-amino-2-[6-chloro-3-(2,3,6-trifluorobenzyl)imidazo[1,5-a]pyridin-1-yl]-5,5-dimethyl-5,7-dihydro-6H-pyrrolo[2,3-d]pyrimidin-6-one (22), 4-amino-2-[6-fluoro-3-(2,3,6-trifluorobenzyl)imidazo[1,5-a]pyridin-1-yl]-5,5-dimethyl-5,7-dihydro-6H-pyrrolo[2,3-d]pyrimidin-6-one (23), 4-amino-2-[6-fluoro-3-(2,3,6-trifluorobenzyl)6-fluoroimidazo[1,5-a]pyridin-1-yl]-5,5-dimethyl-5,7-dihydro-6H-pyrrolo[2,3-d]pyrimidin-6-one (24), 4-amino-5,5-dimethyl-2-[3-(2,4,6-trifluorobenzyl)imidazo[1,5-a]pyridin-1-yl]-5,7-dihydro-6H-pyrrolo[2,3-d]pyrimidin-6-one (25), 4-amino-2-[3-(2-cyclopentylethyl)imidazo[1,5-a]pyridin-1-yl]-5,5-dimethyl-5,7-dihydro-6H-pyrrolo[2,3-d]pyrimidin-6-one (26), disclosed in WO 2010/065275.

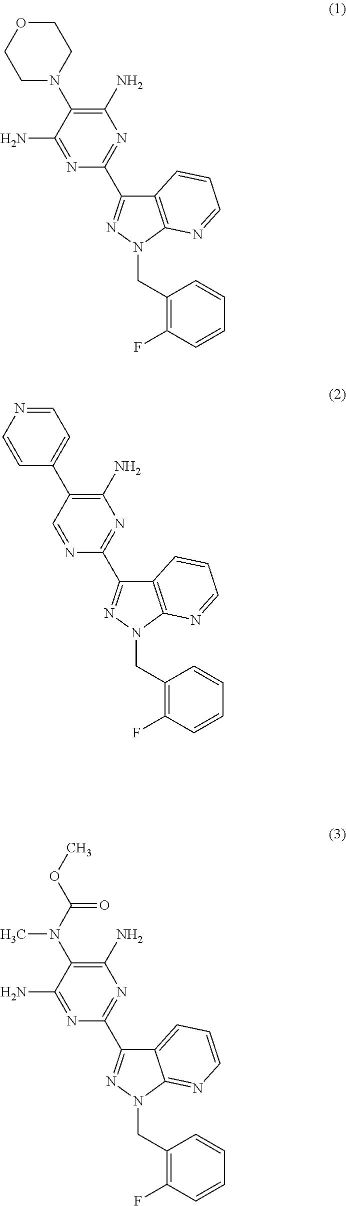 [SMILES: Nc1nc(-c2nn(Cc3ccccc3F)c3ncccc23)nc(N)c1N1CCOCC1 ; Nc1nc(-c2nn(Cc3ccccc3F)c3ncccc23)ncc1-c1ccncc1 ; COC(=O)N(C)c1c(N)nc(-c2nn(Cc3ccccc3F)c3ncccc23)nc1N]

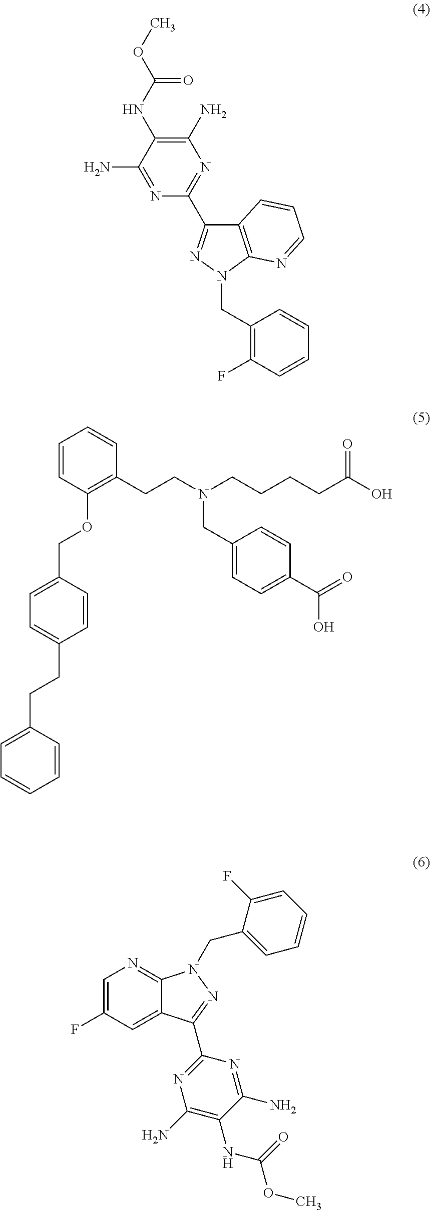 [SMILES: COC(=O)Nc1c(N)nc(-c2nn(Cc3ccccc3F)c3ncccc23)nc1N ; O=C(O)CCCCN(CCc1ccccc1OCc1ccc(CCc2ccccc2)cc1)Cc1ccc(C(=O)O)cc1 ; COC(=O)Nc1c(N)nc(-c2nn(Cc3ccccc3F)c3ncc(F)cc23)nc1N]

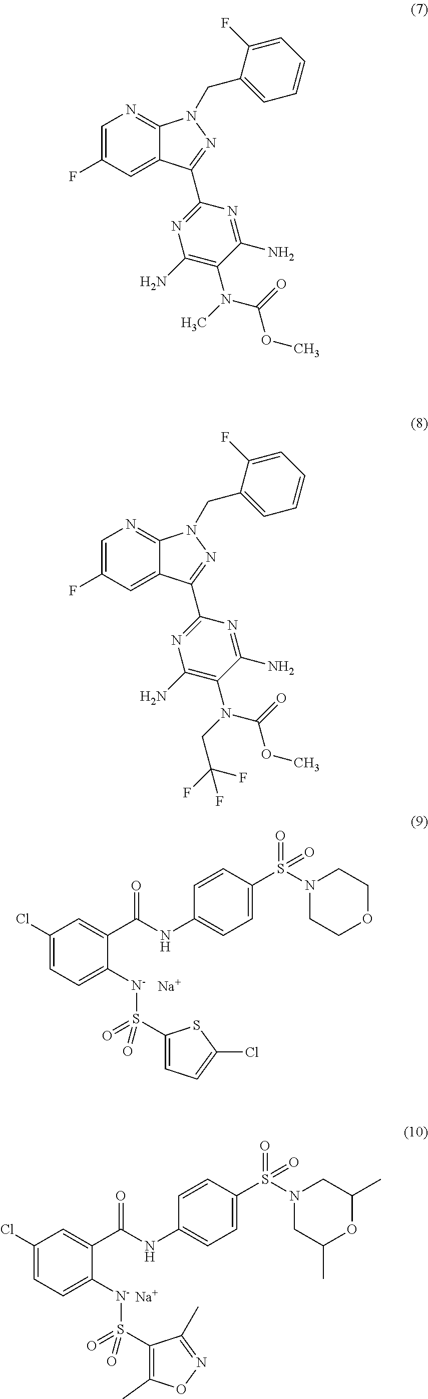 [SMILES: COC(=O)N(C)c1c(N)nc(-c2nn(Cc3ccccc3F)c3ncc(F)cc23)nc1N ; COC(=O)N(CC(F)(F)F)c1c(N)nc(-c2nn(Cc3ccccc3F)c3ncc(F)cc23)nc1N ; O=C(Nc1ccc(S(=O)(=O)N2CCOCC2)cc1)c1cc(Cl)ccc1[N-]S(=O)(=O)c1ccc(Cl)s1 ; [Na+] ; Cc1noc(C)c1S(=O)(=O)[N-]c1ccc(Cl)cc1C(=O)Nc1ccc(S(=O)(=O)N2CC(C)OC(C)C2)cc1 ; [Na+]]

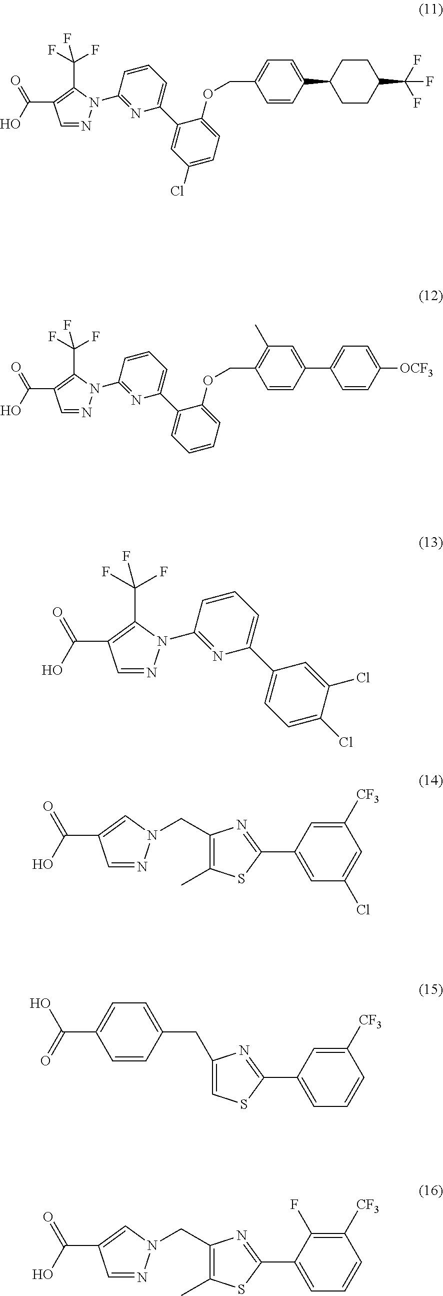

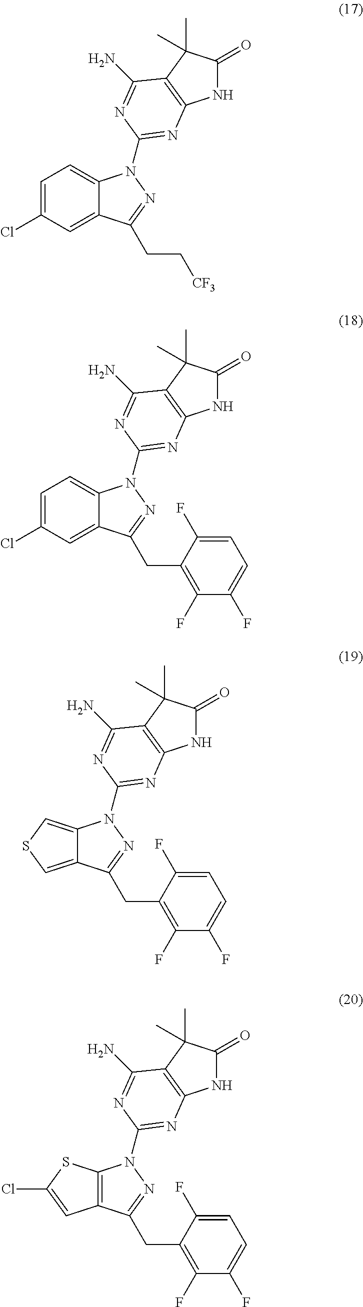

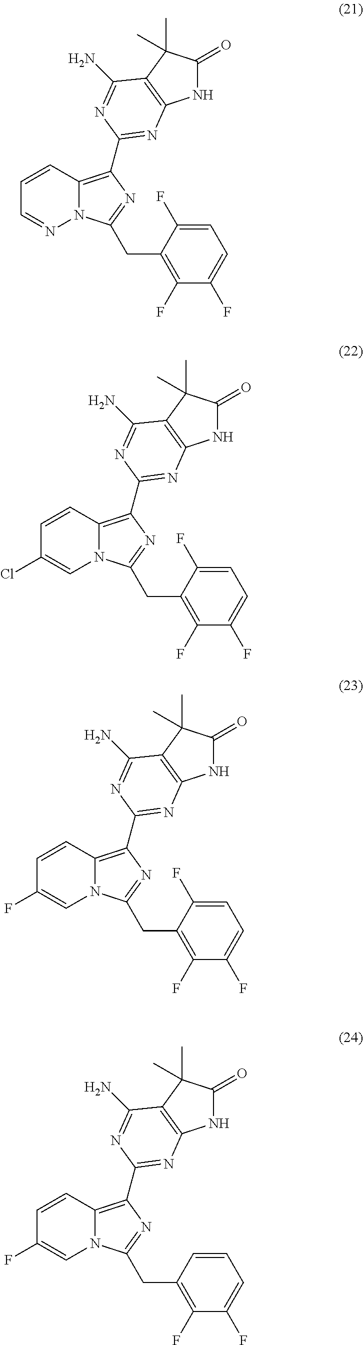

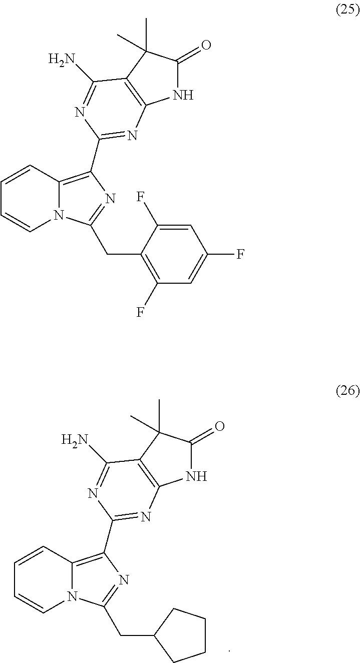 [SMILES: CC1(C)C(=O)Nc2nc(-c3nc(Cc4c(F)cc(F)cc4F)n4ccccc34)nc(N)c21 ; CC1(C)C(=O)Nc2nc(-c3nc(CC4CCCC4)n4ccccc34)nc(N)c21]

Compounds of the formulae (1), (2), (3), (4), (6)-(8) and (17)-(26) are known stimulators of soluble guanylate cyclase. Particular preference is given to the compounds of the formulae (3), (4), (6) and (7).

Compounds of the formulae (5) and (9)-(16) are known activators of soluble guanylate cyclase. Particular preference is given to the compound of the formula (5).

The present invention furthermore provides the combination of stimulators and/or activators of soluble guanylate cyclase with PDE5 inhibitors for use for the treatment of sickle cell anemia and for the preservation of blood substitutes.

The PDE5 inhibitors listed below are suitable for the combination for use for the treatment of sickle cell anemia and preserving blood substitutes:

tadalafil ((6R,12aR)-2,3,6,7,12,12a-hexahydro-2-methyl-6-(3,4-methylenedioxyphenyl)pyrazino (1′,2′:1,6) pyrido(3,4-b)indole-1,4-dione), vardenafil (2-(2-ethoxy-5-(4-ethylpiperazin-1-yl-1-sulfonyl)phenyl)-5-methyl-7-propyl-3H-imidazo(5,1-f)(1,2,4)triazin-4-one), sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), udenafil 5-[2-propyloxy-5-(1-methyl-2-pyrrolidinylethylamidosulfonyl)phenyl]-methyl-3-propyl-1,6-dihydro-7H-pyrazolo(4,3-d)pyrimidin-7-one, dasantafil 7-(3-bromo-4-methoxybenzyl)-1-ethyl-8-[[(1,2)-2-hydroxycyclopentyl]amino]-3-(2-hydroxyethyl)-3,7-dihydro-1-purine-2,6-dione, avanafil 4-{[(3-chloro-4-methoxyphenyl)methyl]amino}-2-[(2S)-2-(hydroxymethyl)pyrrolidin-1-yl]-N-(pyrimidin-2-ylmethyl)pyrimidine-5-carboxamide, mirodenafil, lodenafil, UK 369.003, UK 371.800, SLx 2101 of Surface Logix, LAS 34179, triazolo[1,2-]xanthine, 6-methyl-4-propyl-2-[2-propoxy-5-(4-methylpiperazino)sulfonyl]phenyl or salts, hydrates or hydrates of the salts thereof.

Particular preference is given to combinations of compounds of the formulae (3), (4), (6), (7) and/or (5) with vardenafil and/or sildenafil.

The present invention provides a process for the treatment and/or prophylaxis of sickle cell anemia and for the preservation of blood substitutes using a therapeutically effective amount of one or more compounds of the formulae (1) to (26) according to the invention.

The present invention furthermore provides the use of one or more of the compounds of the formulae (1) to (26) according to the invention for producing a medicament for the treatment and/or prophylaxis of sickle cell anemia and for the preservation of blood substitutes.

The present invention furthermore provides a medicament comprising one or more compounds of the formulae (1) to (26) according to the invention and one or more further active compounds, in particular for the treatment and/or prophylaxis of sickle cell anemia and for the preservation of blood substitutes.

The present invention furthermore provides the use of one or more compounds of the formulae (1) to (26) in combination with one or more of the following PDE5 inhibitors: tadalafil ((6R,12aR)-2,3,6,7,12,12a-hexahydro-2-methyl-6-(3,4-methylenedioxyphenyl)pyrazino (1′,2′:1,6) pyrido(3,4-b)indole-1,4-dione), vardenafil (2-(2-ethoxy-5-(4-ethylpiperazin-1-yl-1-sulfonyl)phenyl)-5-methyl-7-propyl-3H-imidazo(5,1-f)(1,2,4)triazin-4-one), sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), udenafil 5-[2-propyloxy-5-(1-methyl-2-pyrrolidinylethylamidosulfonyl)phenyl]-methyl-3-propyl-1,6-dihydro-7H-pyrazolo(4,3-d)pyrimidin-7-one, dasantafil 7-(3-bromo-4-methoxybenzyl)-1-ethyl-8-[[(1,2)-2-hydroxycyclopentyl]amino]-3-(2-hydroxyethyl)-3,7-dihydro-1-purine-2,6-dione, avanafil 4-{[(3-chloro-4-methoxyphenyl)methyl]amino}-2-[(2S)-2-(hydroxymethyl)pyrrolidin-1-yl]-N-(pyrimidin-2-ylmethyl)pyrimidine-5-carboxamide, mirodenafil, lodenafil, UK 369.003, UK 371.800, SLx 2101 of Surface Logix, LAS 34179, triazolo[1,2-]xanthine, 6-methyl-4-propyl-2-[2-propoxy-5-(4-methylpiperazino)sulfonyl]phenyl or salts, hydrates or hydrates of the salts thereof for the treatment and/or prophylaxis of sickle cell anemia and the preservation of blood substitutes.

The present invention furthermore provides the use of one or more compounds of the formulae (3) to (7) as claimed in claim 1 in combination with vardenafil and/or sildenafil for the treatment and/or prophylaxis of sickle cell anemia and for the preservation of blood substitutes.

The present invention furthermore provides the use of one or more compounds of the formulae (1) to (26) in combination with the PDE5 inhibitors mentioned above for the treatment and/or prophylaxis of traumatized patients receiving a synthetic blood substitute.

The present invention furthermore provides a method for the treatment and/or prophylaxis of sickle cell anemia and for the preservation of blood substitutes using a therapeutically effective amount of one or more compounds of the formulae (1) to (26) according to the invention in combination with the PDE5 inhibitors mentioned above.

The present invention furthermore provides the use of one or more of the compounds of the formulae (1) to (26) according to the invention in combination with the PDE5 inhibitors mentioned above for producing a medicament for the treatment and/or prophylaxis of sickle cell anemia and for the preservation of blood substitutes.

The present invention furthermore provides a medicament comprising one or more compounds of the formulae (1) to (26) according to the invention in combination with the PDE5 inhibitors mentioned above and one or more further active compounds, in particular for the treatment and/or prophylaxis of sickle cell anemia and for the preservation of blood substitutes.

The present invention furthermore provides the use of one or more compounds of the formulae (1) to (26) for the treatment and/or prophylaxis of sickle cell anemia, the administration being by inhalation.

The present invention furthermore provides the use of one or more compounds of the formulae (1) to (26) in combination with the PDE5 inhibitors mentioned above for the treatment and/or prophylaxis of sickle cell anemia, the administration being by inhalation.

In addition, NO- and heme-independent sGC activators were identified, the compound of the formula (5) being a prototype of this class. Common characteristics of these substances are that, in combination with NO, they have only an additive effect on enzyme activation, and that the activation of the oxidized or heme-free enzyme is markedly stronger than that of the heme-containing enzyme [Evgenov et al., ibid.; J. P. Stasch et al., Br. J. Pharmacol. 136 (2002), 773; J. P. Stasch et al., J. Clin. Invest. 116 (2006), 2552]. It is evident from spectroscopic investigations that the compound of the formula (5) displaces the oxidized heme group which, as a result of the weakened iron-histidine bond, is attached only weakly to the sGC. It has also been shown that the characteristic sGC heme binding motive Tyr-x-Ser-x-Arg is imperative both for interaction of the negatively charged propionic acids of the heme group and for the activity of the compound of the formula (5). Against this background, it is assumed that the binding site of the compound of the formula (5) to sGC is identical to the binding site of the heme group [J. P. Stasch et al., J. Clin. Invest. 116 (2006), 2552]. The compounds described in the present invention are likewise capable of activating the heme-free form of the soluble guanylate cyclase. This is also confirmed by the fact that firstly these novel activators display no synergistic effect with NO at the heme-containing enzyme and secondly their action cannot be blocked by the heme-dependent inhibitor of soluble guanylate cyclase, 1H-1,2,4-oxadiazolo[4,3-a]quinoxalin-1-one (ODQ), but is even potentiated by this inhibitor [cf. O. V. Evgenov et al., Nature Rev. Drug Disc. 5 (2006), 755; J. P. Stasch et al., J. Clin. Invest. 116 (2006), 2552], and that these novel activators are to be understood here as sGC activators for the purposes of the present invention. These shall include in particular the sGC activator classes below according to the compounds of the formulae (5) and (9)-(16).

The compounds according to the invention have an unforeseeable useful spectrum of pharmacological and pharmacokinetic activity. In addition to the use for the treatment of sickle cell anemia and the preservation of blood substitutes, the sGC stimulators and/or activators lead to vessel relaxation and/or platelett aggregation inhibition and/or lowering of the blood pressure and/or an increased coronary blood flow and microcirculation in general. They are therefore suitable for the treatment and/or prophylaxis of diseases, preferably cardiovascular diseases, in humans and animals.

Accordingly, the compounds according to the invention are also suitable for the treatment of cardiovascular disorders such as, for example, for the treatment of high blood pressure, for primary and/or secondary prevention, and also for the treatment of heart failure, for the treatment of stable and unstable angina pectoris, pulmonary hypertension, peripheral and cardiac vascular disorders, arrhythmias, for the treatment of thromboembolic disorders and ischemias such as myocardial infarction, stroke, transistoric and ischemic attacks, disturbances of peripheral blood flow, for the prevention of restenoses as after thrombolysis therapies, percutaneous transluminal angioplasties (PTAs), percutaneous transluminal coronary angioplasties (PTCAs) and bypass, and also for the treatment of arteriosclerosis, asthmatic disorders, diseases of the urogenital system such as, for example, prostate hypertrophy, erectile dysfunction, female sexual dysfunction and incontinence.

Moreover, the compounds according to the invention can be used for the treatment of primary and secondary Raynaud's phenomenon, of microcirculation impairments, claudication, peripheral and autonomic neuropathies, diabetic microangiopathies, diabetic nephropathy diabetic retinopathy, diabetic ulcers on the extremities, CREST syndrome, erythematosis, onychomycosis, tinnitus, dizzy spells, sudden deafness and of rheumatic disorders.

The compounds according to the invention are furthermore suitable for the treatment of respiratory distress syndromes and chronic-obstructive pulmonary disease (COPD), of acute and chronic kidney failure and for promoting wound healing.

The compounds according to the invention are furthermore also suitable for controlling cerebral blood flow and thus represent effective agents for controlling migraine. They are also suitable for the prophylaxis and control of sequelae of cerebral infarct (Apoplexia cerebri) such as stroke, cerebral ischemias and skull-brain trauma. The compounds according to the invention can likewise be employed for controlling states of pain.

In addition, the compounds according to the invention can also be employed for the treatment and/or prevention of micro- and macrovascular damage (vasculitis), reperfusion damage, arterial and venous thromboses, edemas, neoplastic disorders (skin cancer, liposarcomas, carcinomas of the gastrointestinal tract, of the liver, of the pancreas, of the lung, of the kidney, of the ureter, of the prostate and of the genital tract), of disorders of the central nervous system and neurodegenerative disorders (stroke, Alzheimer's disease, Parkinson's disease, dementia, epilepsy, depressions, multiple sclerosis), of inflammatory disorders, immune disorders (Crohn's disease, ulcerative colitis, lupus erythematosus, rheumatoid arthritis, asthma), kidney disorders (glomerulonephritis), thyroid disorders (hyperthyreosis), disorders of the pancreas (pancreatitis), liver fibrosis, skin disorders (psoriasis, acne, eczema, neurodermitis, dermatitis, keratitis, formation of scars, formation of warts, chilblains), viral disorders (HPV, HCMV, HIV), cachexia, osteoporosis, gout, incontinence, and also for wound healing and angiogenesis.

The present invention furthermore provides the use of the compounds according to the invention for the treatment and/or prophylaxis of disorders, preferably of thromboembolic disorders and/or thromboembolic complications.

“Thromboembolic disorders” in the sense of the present invention include in particular disorders such as ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, deep venous thromboses and renal vein thromboses, transitory ischemic attacks and also thrombotic and thromboembolic stroke and pulmonary hypertension.

Accordingly, the substances are also suitable for the prevention and treatment of cardiogenic thromboembolisms, such as, for example, brain ischemias, stroke and systemic thromboembolisms and ischemias, in patients with acute, intermittent or persistent cardiac arrhythmias, such as, for example, atrial fibrillation, and those undergoing cardioversion, furthermore in patients with heart valve disorders or with intravasal objects, such as, for example, artificial heart valves, catheters, intraaortic balloon counterpulsation and pacemaker probes. In addition, the compounds according to the invention are suitable for the treatment of disseminated intravasal coagulation (DIC).

Thromboembolic complications furthermore occur in microangiopathic hemolytic anemias, sickle cell anemia, thalassemia, inherited forms of spherocytosis, eliptocytosis and ovalcytosis, malaria, Moskowitz syndrome, hemolytic uremia syndrome, extracorporal circulation such as, for example, hemodialysis, hemofiltration, ventricular assist devices and artificial heart, and also heart valve protheses, blood transfusions, for cardiopulmonary bypass and organ transplantations (for example lung, heart, kidney, liver), hemolysis owing to intravascular devices or owing to hemodialysis, and also when using synthetic Hb-based oxygen carriers.

The compounds according to the invention are also particularly suitable for the primary and/or secondary prevention and for the treatment of heart failure.

In the context of the present invention, the term heart failure also includes more specific or related types of disease, such as right heart failure, left heart failure, global failure, ischemic cardiomyopathy, dilated cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and diastolic and systolic heart failure.

The present invention further provides for the use of the compounds according to the invention for the treatment and/or prophylaxis of disorders, in particular the disorders mentioned above.

Furthermore, the present invention is also suitable for the treatment of traumatized patients receiving a synthetic blood substitute [Weisskopf 2010].

The present invention further provides for the use of the compounds according to the invention for producing a medicament for the treatment and/or prophylaxis of disorders, in particular the disorders mentioned above.

The present invention further provides a method for the treatment and/or prophylaxis of disorders, especially the disorders mentioned above, using a therapeutically effective amount of a compound according to the invention.

The present invention further provides for the use of the compounds according to the invention for producing a medicament for the treatment and/or prophylaxis of sickle cell anemia and for the preservation of blood substitutes.

The present invention further provides a method for the treatment and/or prophylaxis of sickle cell anemia and for the preservation of blood substitutes, using a therapeutically effective amount of a compound according to the invention.

The inventive compounds can be employed alone or, if required, in combination with other active ingredients.

The present invention further provides medicaments comprising a compound according to the invention and one or more further active compounds, especially for the treatment and/or prophylaxis of the disorders mentioned above. Suitable active ingredients for combination are, by way of example and by way of preference: active ingredients which modulate lipid metabolism, antidiabetics, hypotensive agents, perfusion-enhancing and/or antithrombotic agents, and also antioxidants, aldosterone- and mineralocorticoid receptor antagonists, vasopressin receptor antagonists, organic nitrates and NO donors, IP receptor agonists, positively inotropically active compounds, ACE inhibitors, cGMP- and cAMP-modulating compounds, inhibitors of neutrophile elastase, signal transduction cascade-inhibiting compounds, compounds that modulate the energy metabolism of the heart, chemokine receptor antagonists, p38 kinase inhibitors, NPY agonists, orexin agonists, anorectics, PAF-AH inhibitors, antiphlogistics (COX inhibitors, LTB₄ receptor antagonists, inhibitors of LTB₄ synthesis), analgesics (aspirin), antidepressants and other psychopharmaceuticals.

The present invention provides in particular combinations of at least one of the compounds according to the invention and at least one lipid metabolism-modifying active compound, antidiabetic, hypotensive active compound and/or agent having antithrombotic action.

The compounds according to the invention can preferably be combined with one or more
lipid metabolism-modulating active ingredients, by way of example and by way of preference from the group of the HMG-CoA reductase inhibitors from the class of the statins such as, by way of example and by way of preference, lovastatin, simvastatin, pravastatin, fluvastatin, atorvastatin, rosuvastatin, cerivastatin or pitavastatin, inhibitors of HMG-CoA reductase expression, squalene synthesis inhibitors such as, by way of example and by way of preference, BMS-188494 or TAK-475, ACAT inhibitors such as, by way of example and by way of preference, melinamide, pactimibe, eflucimibe or SMP-797, LDL receptor inductors, cholesterol absorption inhibitors such as, by way of example and by way of preference, ezetimibe, tiqueside or pamaqueside, polymeric bile acid adsorbers such as, by way of example and by way of preference, cholestyramine, colestipol, colesolvam, CholestaGel or colestimide, bile acid reabsorption inhibitors such as, by way of example and by way of preference, ASBT(=IBAT) inhibitors such as AZD-7806, S-8921, AK-105, BARI=1741, SC-435 or SC-635, MTP inhibitors such as, by way of example and by way of preference, implitapide or JTT-130, lipase inhibitors such as, by way of example and by way of preference, orlistat, LpL activators, fibrates, niacin, CETP inhibitors such as, by way of example and by way of preference, torcetrapib, JTT-705 or CETP vaccine (Avant), PPAR-β and/or PPAR-δ agonists such as, by way of example and by way of preference, pioglitazone or rosiglitazone and/or GW-501516, RXR modulators, FXR modulators, LXR modulators, thyroid hormones and/or thyroid mimetics such as, by way of example and by way of preference, D-thyroxine or 3,5,3′-triiodothyronine (T3), ATP citrate lyase inhibitors, Lp(a) antagonists, cannabinoid receptor 1-antagonists such as, by way of example and by way of preference, rimonabant or SR-147778, leptin receptor agonists, bombesin receptor agonists, histamine receptor agonists, agonists of the niacin receptor such as, by way of example and by way of preference, niacin, acipimox, acifran or radecol, and the antioxidants/radical scavengers such as, by way of example and by way of preference, probucol, AGI-1067, BO-653 or AEOL-10150,
   antidiabetics mentioned in Die Rote Liste 2009/I, chapter 12. Antidiabetics are preferably understood as meaning insulin and insulin derivatives and also orally effective hypoglycemically active compounds. Here, insulin and insulin derivatives include both insulins of animal, human or biotechnological origin and mixtures thereof. The orally effective hypoglycemically active compounds preferably include sulfonylureas, biguanides, meglitinide derivatives, glucosidase inhibitors and PPAR-gamma agonists, and also, by way of example and by way of preference, those from the group of the sulfonylureas such as, by way of example and by way of preference, tolbutamide, glibenclamide, glimepiride, glipizide or gliclazide, biguanides such as, by way of example and by way of preference, metformin, meglitinide derivatives such as, by way of example and by way of preference, repaglinide or nateglinide, glucosidase inhibitors such as, by way of example and by way of preference, miglitol or acarbose, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and potassium channel openers such as, for example, those disclosed in WO 97/26265 and WO 99/03861;
hypotensive active compounds, by way of example and by way of preference from the group of the calcium antagonists such as, by way of example and by way of preference, nifedipine, amlodipine, verapamil or diltiazem, angiotensin AII antagonists such as, by way of example and by way of preference, losartan, valsartan, candesartan, embusartan or telmisartan, ACE inhibitors such as, by way of example and by way of preference, enalapril, captopril, ramipril, delapril, fosinopril, quinopril, perindopril or trandopril, beta receptor blockers such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol, alpha receptor blockers such as, by way of example and by way of preference, prazosin, ECE inhibitors, rho-kinase inhibitors and of the vasopeptidase inhibitors, and also of the diuretics such as, by way of example and by way of preference, a loop diuretic such as furosemide, bumetanide or torsemide, or a thiazide or thiazide-like diuretic such as chlorothiazide or hydrochlorothiazide or A1 antagonists such as rolofylline, tonopofylline and SLV-320;
agents which lower the sympathetic tone such as, by way of example and by way of preference, reserpin, clonidine or alpha-methyldopa, or in combination with a potassium channel agonist such as, by way of example and by way of preference, minoxidil, diazoxide, dihydralazine or hydralazine,
agents with antithrombotic action such as, by way of example and by way of preference, from the group of the platelett aggregation inhibitors such as, by way of example and by way of preference, aspirin, clopidogrel, ticlopidine, cilostazol or dipyridamole, or of the anticoagulants such as thrombin inhibitors such as, by way of example and by way of preference, ximelagatran, melagatran, bivalirudin or clexane, a GPIIb/IIIa antagonist such as, by way of example and by way of preference, tirofiban or abciximab, a factor Xa inhibitor such as, by way of example and by way of preference, rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428, with heparin or a low molecular weight (LMW) heparin derivative or with a vitamin K antagonist such as, by way of example and by way of preference, coumarin,
aldosterone and mineralocorticoid receptor antagonists such as, by way of example and by way of preference, spironolactone or eplerenone;
vasopressin receptor antagonists such as, by way of example and by way of preference, conivaptan, tolvaptan, lixivaptan or SR-121463;
organic nitrates and NO donors such as, by way of example and by way of preference, sodium nitroprusside, nitroglycerol, isosorbide mononitrate, isosorbide dinitrate, molsidomine or SIN-1, or in combination with inhalative NO;
IP receptor agonsists, preferred examples being iloprost, treprostinil, beraprost and NS-304;
compounds having a positive inotropic effect, preferred examples being cardiac glycosides (digoxin), beta-adrenergic and dopaminergic agonists such as isoproterenol, adrenaline, noradrenaline, dopamine and dobutamine;
calcium sensitizers, a preferred example being levosimendan;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil, and PDE 3 inhibitors such as milrinone;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
NO-independent but heme-dependent stimulators of guanylate cyclase, such as especially the compounds described in WO 00/06568, WO 00/06569, WO 02/42301 and WO 03/095451;
NO- and heme-independent activators of guanylate cyclase, such as especially the compounds described in WO 01/19355, WO 01/19776, WO 01/19778, WO 01/19780, WO 02/070462 and WO 02/070510;
inhibitors of human neutrophil elastase (HNE), for example sivelestat and DX-890 (Reltran);
compounds which inhibit the signal transduction cascade, for example tyrosine kinase inhibitors and multikinase inhibitors, especially sorafenib, imatinib, gefitinib and erlotinib; and/or
compounds which influence the energy metabolism of the heart, such as, for example, etomoxir, dichloroacetate, ranolazine and trimetazidine.

In the context of the present invention, particular preference is given to combinations comprising at least one of the compounds according to the invention and one or more further active compounds selected from the group consisting of HMG-CoA reductase inhibitors (statins), diuretics, antidiabetics, beta-receptor blockers, organic nitrates and NO donors, ACE inhibitors, angiotensin AII antagonists, aldosterone and mineralocorticoid receptor antagonists, vasopressin receptor antagonists, platelett aggregation inhibitors and anticoagulants, and also their use for the treatment and/or prevention of the disorders mentioned above.

The compounds according to the invention may act systemically and/or locally. For this purpose, they can be administered in a suitable manner, for example by the oral, parenteral, pulmonal, nasal, sublingual, lingual, buccal, rectal, dermal, transdermal, conjunctival, otic route, or as an implant or stent.

The compounds according to the invention can be administered in administration forms suitable for these administration routes.

Suitable administration forms for oral administration are those which function according to the prior art and deliver the compounds according to the invention rapidly and/or in modified fashion, and which contain the compounds according to the invention in crystalline and/or amorphized and/or dissolved form, for example tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. intramuscularly, subcutaneously, intracutaneously, percutaneously or intraperitoneally). Suitable administration forms for parenteral administration include injection and infusion formulations in the form of solutions, suspensions, emulsions, lyophilizates or sterile powders.

Oral administration is preferred.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

Administration by inhalation is likewise preferred.

The compounds according to the invention can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavor and/or odor correctants.

The present invention further provides medicaments comprising at least one inventive compound, preferably together with one or more inert nontoxic pharmaceutically suitable excipients, and the use thereof for the treatment of sickle cell anemia and for preserving blood substitutes.

In the case of parenteral administration, it has generally been found to be advantageous to administer amounts of about 5 to 100 mg every 24 hours to achieve effective results. In the case of oral administration, the amount is about 5 to 250 mg every 24 hours.

It may nevertheless be necessary where appropriate to deviate from the stated amounts, specifically as a function of the body weight, route of administration, individual response to the active ingredient, nature of the preparation and time or interval over which administration takes place.

The percentages in the tests and examples which follow are, unless indicated otherwise, percentages by weight; parts are parts by weight. Solvent ratios, dilution ratios and concentration figures for liquid/liquid solutions are each based on volume. “w/v” means “weight/volume”. For example, “10% w/v” means: 100 ml of solution or suspension comprise 10 g of substance.

### EXPERIMENTAL PART

### A. Examples

### Abbreviations and Acronyms

aq. aqueous solution
calc. calculated
DCI direct chemical ionization (in MS)
DMF dimethylformamide
DMSO dimethyl sulfoxide
eq. equivalent(s)
ESI electrospray ionization (in MS)
Et ethyl
h hour(s)
HPLC high-pressure, high-performance liquid chromatography
HRMS high-resolution mass spectrometry
conc. concentrated
LC/MS liquid chromatography-coupled mass spectrometry
LiHMDS lithium hexamethyldisilazide
Me methyl
min minute(s)
MS mass spectrometry
NMR nuclear magnetic resonance spectroscopy
Pd₂dba₃ tris(dibenzylideneacetone)dipalladium
Ph phenyl
RT room temperature
Rₜ retention time (in HPLC)
THF tetrahydrofuran
UV ultraviolet spectrometry
v/v ratio by volume (of a solution)
XPHOS dicyclohexyl(2′,4′,6′-triisopropylbiphenyl-2-yl)phosphine

### LC/MS Methods:

### Method 1:

MS instrument type: Waters ZQ; HPLC instrument type: Agilent 1100 Series; UV DAD; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.1 min 100% A (flow rate 2.5 ml/min); oven: 55° C.; flow rate: 2 ml/min; UV detection: 210 nm.

### Method 2:

Instrument: Waters ACQUITY SQD UPLC System; column: Waters Acquity UPLC HSS T3 1.8μ50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

### Starting Materials and Intermediates

### Example 1A

### 2,6-Dichloro-5-fluoronicotinamide

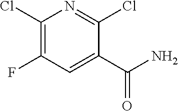 [SMILES: NC(=O)c1cc(F)c(Cl)nc1Cl]

A suspension of 25 g (130.90 mmol) of 2,6-dichloro-5-fluoro-3-cyanopyridine in conc. sulfuric acid (125 ml) was stirred at 60-65° C. for 1 h. After cooling to RT, the contents of the flask were poured into ice-water and extracted three times with ethyl acetate (100 ml each time). The combined organic phases were washed with water (100 ml) and then with saturated aqueous sodium hydrogen carbonate solution (100 ml), dried and concentrated on a rotary evaporator. The material obtained was dried under a high vacuum.

Yield: 24.5 g (90% of theory)

¹H-NMR (400 MHz, DMSO-d₆): δ=7.95 (br s, 1H), 8.11 (br s, 1H), 8.24 (d, 1H).

### Example 2A

### 2-Chloro-5-fluoronicotinamide

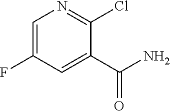 [SMILES: NC(=O)c1cc(F)cnc1Cl]

A suspension of 21.9 g (335.35 mmol) of zinc in methanol (207 ml) was admixed at RT with 44 g (210.58 mmol) of 2,6-dichloro-5-fluoronicotinamide. Acetic acid (18.5 ml) was then added, and the mixture was heated with stirring at reflux for 24 h. The contents of the flask were then decanted from the zinc, and ethyl acetate (414 ml) and saturated aqueous sodium hydrogen carbonate solution (414 ml) were added, followed by intense extractive stirring. Subsequently the reaction mixture was filtered with suction through kieselguhr and the filter product was washed three times with ethyl acetate (517 ml each time). The organic phase was separated off and the aqueous phase was washed with ethyl acetate (258 ml). The combined organic phases were washed once with saturated aqueous sodium hydrogen carbonate solution (414 ml), dried and concentrated under reduced pressure. Dichloromethane (388 ml) was added to the crystals obtained in this manner, and the mixture was subjected to extractive stirring for 20 min. The mixture was once more filtered off with suction, washed with diethyl ether and sucked dry.

Yield: 20.2 g (53% of theory)

¹H-NMR (400 MHz, DMSO-d₆): δ=7.87 (br s, 1H), 7.99 (dd, 1H), 8.10 (br s, 1H), 8.52 (d, 1H).

### Example 3A

### 2-Chloro-5-fluoronicotinonitrile

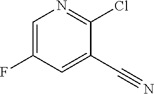

81.2 ml (582.25 mmol) of triethylamine were added to a suspension of 46.2 g (264.66 mmol) of 2-chloro-5-fluoronicotinamide in dichloromethane (783 ml), and the mixture was cooled to 0° C. Then, with stirring, 41.12 ml (291.13 mmol) of trifluoroacetic anhydride were added slowly dropwise and the mixture was stirred at 0° C. for 1.5 h. The reaction solution was subsequently washed twice with saturated aqueous sodium bicarbonate solution (391 ml each time), dried and concentrated under reduced pressure.

Yield: 42.1 g (90% of theory)

¹H-NMR (400 MHz, DMSO-d₆): δ=8.66 (dd, 1H), 8.82 (d, 1H).

### Example 4A

### 5-Fluoro-1H-pyrazolo[3,4-b]pyridine-3-amine

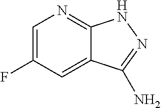 [SMILES: Nc1n[nH]c2ncc(F)cc12]

A suspension of 38.5 g (245.93 mmol) of 2-chloro-5-fluoronicotinonitrile was initially charged in 1,2-ethanediol (380 ml), and hydrazine hydrate (119.6 ml, 2.459 mol) was then added. The mixture was heated under reflux with stirring for 4 h. The product precipitated in the course of cooling. Water (380 ml) was added to the yellow crystals, and the mixture was subjected to extractive stirring at RT for 10 min. The suspension was then filtered with suction over a frit, and the filter product was washed with water (200 ml) and with −10° C. cold THF (200 ml). The residue was dried under a high vacuum over phosphorus pentoxide.

Yield: 22.8 g (61% of theory)

¹H-NMR (400 MHz, DMSO-d₆): δ=5.54 (s, 2H), 7.96 (dd, 1H), 8.38 (m, 1H), 12.07 (m, 1H).

### Example 5A

### 5-Fluoro-3-iodo-1H-pyrazolo[3,4-b]pyridine

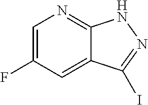 [SMILES: Fc1cnc2[nH]nc(I)c2c1]

10 g (65.75 mmol) of 5-fluoro-1H-pyrazolo[3,4-b]pyridine-3-amine were initially charged in THF (329 ml), and the mixture was cooled to 0° C. 16.65 ml (131.46 mmol) of boron trifluoride diethyl ether complex were then added slowly. The reaction mixture was cooled further to −10° C. A solution of 10.01 g (85.45 mmol) of isopentyl nitrite in THF (24.39 ml) was then added slowly, and the mixture was stirred for a further 30 min. The mixture was diluted with cold diethyl ether (329 ml) and the resulting solid was isolated by filtration. A little at a time, the diazonium salt thus prepared was added to a cold (0° C.) solution of 12.81 g (85.45 mmol) of sodium iodide in acetone (329 ml), and the mixture was stirred at RT for 30 min. The reaction mixture was poured into ice-water (1.8 l) and extracted twice with ethyl acetate (487 ml each time). The collected organic phases were washed with saturated aqueous sodium chloride solution (244 ml), dried, filtered and concentrated. This gave 12.1 g (86% purity, 60% of theory) of the desired compound in the form of a brown solid. The crude product was reacted without further purification.

LC-MS (method 1): Rₜ=1.68 min; MS (ESIpos): m/z=264 (M+H)⁺

### Example 6A

### 5-Fluoro-1-(2-fluorobenzyl)-3-iodo-1H-pyrazolo[3,4-b]pyridine

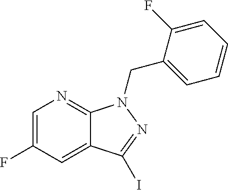 [SMILES: Fc1cnc2c(c1)c(I)nn2Cc1ccccc1F]

141 g (462.11 mmol) of the compound from Example 5A were introduced into DMF (2538 ml), and 96.09 g (508.32 mmol) of 2-fluorobenzyl bromide and 165.62 g (508.32 mmol) of caesium carbonate were then added. The mixture was stirred at RT for two hours. The reaction mixture was then poured into saturated aqueous sodium chloride solution (13 670 ml) and extracted twice with ethyl acetate (5858 ml). The collected organic phases were washed with saturated aqueous sodium chloride solution (3905 ml), dried, filtered and concentrated. The residue was chromatographed on silica gel (mobile phase: petroleum ether/ethyl acetate 97:3) and the product fractions were concentrated. The resulting solid was dissolved in dichloromethane and washed once with saturated aqueous sodium thiosulfate solution (500 ml) and then with saturated aqueous sodium chloride solution (500 ml). The product was concentrated to dryness and the residue was suspended in diethyl ether, isolated by filtration with suction and dried under high vacuum. This gave 106.6 g (62% of theory) of the desired compound.

LC-MS (method 1): Rₜ=2.57 min

MS (ESIpos): m/z=372 (M+H)⁺

¹H-NMR (400 MHz, DMSO-d₆): δ=5.73 (s, 2H), 7.13-7.26 (m, 3H), 7.33-7.41 (m, 1H), 7.94 (dd, 1H), 8.69-8.73 (m, 1H).

### Example 7A

### 2-[5-Fluoro-1-(2-fluorobenzyl)-1H-pyrazolo[3, 4-1)]pyridin-3-yl]-5-nitropyrimidine-4,6-diamine

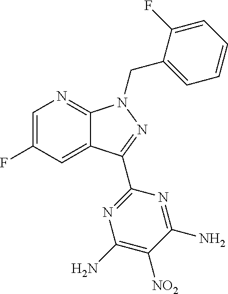 [SMILES: Nc1nc(-c2nn(Cc3ccccc3F)c3ncc(F)cc23)nc(N)c1[N+](=O)[O-]]

Under argon, 860 mg (2.32 mmol) of the compound from Example 6A were introduced into 1,4-dioxane (86 ml), and the reaction mixture was flushed with argon for 10 min. Then 3.51 ml (6.95 mmol) of hexabutylditin and 483 mg (2.55 mmol) of 2-chloro-5-nitropyrimidine-4,6-diamine (prepared by the method of Helvetica Chimica Acta (1951), 34, 835-40) were added. Subsequently 860 mg (0.744 mmol) of tetrakis(triphenylphosphine)palladium(0) were added and the reaction mixture was heated at reflux overnight. The mixture was then cooled to RT, water was added and the mixture was extracted twice with ethyl acetate. The collected organic phases were dried over sodium sulfate, filtered and concentrated. The residue was subjected to extractive stirring in ethyl acetate, and the solid was isolated by filtration and dried under high vacuum. This gave 355 mg (62% purity, 24% of theory) of the desired compound. The crude product was reacted without further purification.

LC-MS (method 2): Rₜ=1.03 min

MS (ESIpos): m/z=399 (M+H)⁺

### Example 8A

### 5-Fluoro-1-(2-fluorobenzyl)-1H-pyrazolo[3,4-b]pyridine-3-carbonitrile

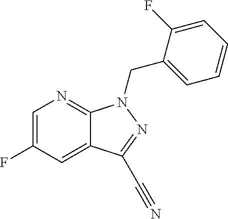 [SMILES: N#Cc1nn(Cc2ccccc2F)c2ncc(F)cc12]

A suspension of 16.03 g (43.19 mmol) of 5-fluoro-1-(2-fluorobenzyl)-3-iodo-1H-pyrazolo[3,4-b]pyridine (Example 6A) and 4.25 g (47.51 mmol) of copper cyanide was initially charged in DMSO (120 ml) and stirred at 150° C. for 2 h. After cooling, the contents of the flask were cooled to about 40° C. and poured into a solution of conc. aqueous ammonia (90 ml) and water (500 ml), ethyl acetate (200 ml) was added and the mixture was subjected to brief extractive stirring. The aqueous phase was separated off and extracted two more times with ethyl acetate (200 ml each time). The combined organic phases were washed twice with 10% strength aqueous sodium chloride solution (100 ml each time), dried and concentrated under reduced pressure. The crude product was reacted without further purification.

Yield: 11.1 g (91% of theory)

¹H-NMR (400 MHz, DMSO-d₆): δ=5.87 (s, 2H), 7.17-7.42 (m, 4H), 8.52 (dd, 1H), 8.87 (dd, 1H).

### Example 9A

### 5-Fluoro-1-(2-fluorobenzyl)-1H-pyrazolo[3,4-b]pyridine-3-carboximidamide acetate

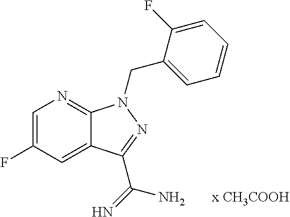 [SMILES: N=C(N)c1nn(Cc2ccccc2F)c2ncc(F)cc12]

11.1 g (41.07 mmol) of 5-fluoro-1-(2-fluorobenzyl)-1H-pyrazolo[3,4-b]pyridine-3-carbonitrile (Example 8A) were added to 2.22 g (41.07 mmol) of sodium methoxide in methanol (270 ml), and the mixture was stirred at RT for 2 h. 2.64 g (49.29 mmol) of ammonium chloride and acetic acid (9.17 ml) were then added, and the mixture was heated at reflux overnight. It was then concentrated to dryness and the residue was taken up in water (100 ml) and ethyl acetate (100 ml) and adjusted to a pH of 10 using 2N aqueous sodium hydroxide solution. The mixture was stirred intensively at RT for about 1 h. The resulting suspension was filtered with suction and the filter product was washed with ethyl acetate (100 ml), with water (100 ml) and once more with ethyl acetate (100 ml). The residue was dried under high vacuum over phosphorus pentoxide.

Yield: 9.6 g (78% of theory)

MS (ESIpos): m/z=288 (M+H)⁺

¹H-NMR (400 MHz, DMSO-d₆): δ=1.85 (s, 3H), 5.80 (s, 2H), 7.14-7.25 (m, 3H), 7.36 (m, 1H), 8.42 (dd, 1H), 8.72 (dd, 1H).

### Example 10A

### 2-[5-Fluoro-1-(2-fluorobenzyl)-1H-pyrazolo[3,4-b]pyridin-3-yl]-5-[(E)-phenyldiazenyl]pyrimidine-4,6-diamine

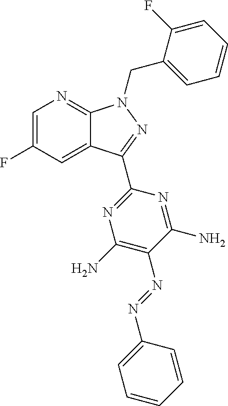 [SMILES: Nc1nc(-c2nn(Cc3ccccc3F)c3ncc(F)cc23)nc(N)c1/N=N/c1ccccc1]

With stirring, 3.85 g (41.34 mmol) of aniline were added to water (40 ml) and conc. hydrochloric acid (7.07 ml), and this mixture was cooled to 0° C. A solution of 2.85 g (41.34 mmol) of sodium nitrite in water (21 ml) was then added dropwise at between 0° C. and 5° C., followed by stirring at 0° C. for 15 min. Thereafter, at 0° C., a solution of 4.28 g (52.25 mmol) of sodium acetate in water (19 ml) was added rapidly dropwise, and then, with thorough stirring, a solution of 2.73 g (41.34 mmol) of malononitrile in ethanol (10 ml) was added dropwise. After 2 h at 0° C., the resulting precipitate was isolated by filtration with suction and washed three times with water (50 ml each time) and with petroleum ether (50 ml). The residue, still moist, was dissolved in DMF (46 ml) and added dropwise at precisely 85° C. to a solution of 9.5 g (33.07 mmol) of 5-fluoro-1-(2-fluorobenzyl)-1H-pyrazolo[3,4-b]pyridine-3-carboximidamide acetate (Example 9A) in DMF (46 ml) and triethylamine (5.76 ml). The mixture was then stirred at 100° C. for 4 h and left to cool to RT overnight. The mixture was poured into water (480 ml) and subjected to extractive stirring at RT for 1 h. After the precipitate had been isolated by filtration with suction, it was washed twice with water (100 ml each time) and twice with methanol (50 ml each time) and then dried under a high vacuum.

Yield: 9.6 g (59% of theory)

LC-MS (method 2): Rₜ=1.21 min

MS (ESIpos): m/z=458 (M+H)⁺

### Example 11A

### 2-[5-Fluoro-1-(2-fluorobenzyl)-1H-pyrazolo[3,4-b]pyridin-3-yl]pyrimidine-4,5,6-triamine

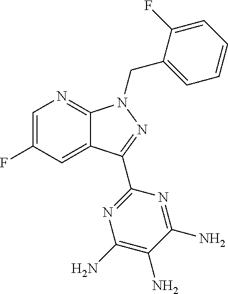 [SMILES: Nc1nc(-c2nn(Cc3ccccc3F)c3ncc(F)cc23)nc(N)c1N]

### Variant A:

Preparation starting from Example 7A:

In pyridine (30 ml), 378 mg (0.949 mmol) of the compound from Example 7A were introduced and then 143 mg (0.135 mmol) of palladium (10% on carbon) were added. The mixture was hydrogenated overnight at RT under standard hydrogen pressure. The suspension was then filtered through kieselguhr and the filtercake was washed with ethanol. The filtrate was concentrated and yielded 233 mg (81% purity, 51% of theory) of the desired compound, which were reacted without further purification.

### Variant B:

Preparation starting from Example 10A:

In DMF (800 ml), 39.23 g (85.75 mmol) of the compound from Example 10A were introduced and then 4 g of palladium (10% on carbon) were added. The mixture was hydrogenated with stirring overnight under standard hydrogen pressure. The batch was filtered over kieselguhr and the filter product was washed with a little DMF and then with a little methanol, and concentrated to dryness. The residue was admixed with ethyl acetate and stirred vigorously, and the precipitate was filtered off with suction, washed with ethyl acetate and diisopropyl ether and dried under a high vacuum over Sicapent.

Yield: 31.7 g (100% of theory)

LC-MS (method 2): Rt=081 min

MS (ESIpos): m/z=369 (M+H)⁺

### Working Examples

### Example 1

### Methyl {4,6-diamino-2-[5-fluoro-1-(2-fluorobenzyl)-1H-pyrazolo[3,4-b]pyridin-3-yl]pyrimidin-5-yl}carbamate

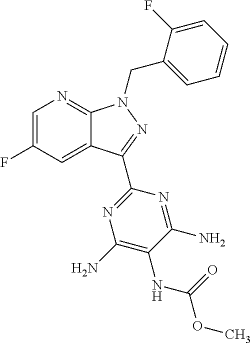 [SMILES: COC(=O)Nc1c(N)nc(-c2nn(Cc3ccccc3F)c3ncc(F)cc23)nc1N]

In pyridine (600 ml), 31.75 g (86.20 mmol) of the compound from Example 11A were introduced under argon and cooled to 0° C. Then a solution of 6.66 ml (86.20 mmol) of methyl chloroformate in dichloromethane (10 ml) was added dropwise and the mixture was stirred at 0° C. for 1 h. Thereafter the reaction mixture was brought to RT, concentrated under reduced pressure and co-distilled repeatedly with toluene. The residue was stirred with water/ethanol and then filtered off with suction on a frit, after which it was washed with ethanol and ethyl acetate. Subsequently the residue was again stirred with diethyl ether, isolated by filtration with suction and then dried under a high vacuum.

Yield: 24.24 g (65% of theory)

LC-MS (method 2): Rₜ=0.79 min

MS (ESIpos): m/z=427 (M+H)+

¹H-NMR (400 MHz, DMSO-d₆): δ=3.62 (br. s, 3H), 5.79 (s, 2H), 6.22 (br. s, 4H), 7.10-7.19 (m, 2H), 7.19-7.26 (m, 1H), 7.32-7.40 (m, 1H), 7.67 (br. s, 0.2H), 7.99 (br. s, 0.8H), 8.66 (m, 1H), 8.89 (d, 1H).

### Example 2

### Methyl {4,6-diamino-2-[5-fluoro-1-(2-fluorobenzyl)-1H-pyrazolo[3,4-b]pyridin-3-yl]pyrimidin-5-yl}methylcarbamate

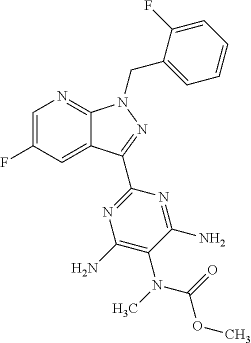 [SMILES: COC(=O)N(C)c1c(N)nc(-c2nn(Cc3ccccc3F)c3ncc(F)cc23)nc1N]

A quantity of 200 mg (0.469 mmol) of methyl {4,6-diamino-2-[5-fluoro-1-(2-fluorobenzyl)-1H-pyrazolo[3,4-b]pyridin-3-yl]pyrimidin-5-yl}carbamate (Example 1) was introduced in THF (5 ml) at 0° C. Then 0.704 ml (0.704 mmol) of lithium hexamethyldisilazane solution (1M in THF) was added and the mixture was stirred at this temperature for 20 min. Subsequently 43.8 μl (0.704 mmol) of iodomethane were added and the mixture was warmed to RT. After 1 h at this temperature, reaction was terminated with water (1 ml) and the reaction mixture was concentrated, the residue being separated by means of preparative RP-HPLC (water (+0.05% formic acid)-acetonitrile gradient).

Yield: 90 mg (44% of theory)

LC-MS (method 2): Rₜ=0.85 min

MS (ESIpos): m/z=441 (M+H)⁺

¹H-NMR (400 MHz, DMSO-d₆): δ=3.00 (s, 3H), 3.53 (s, 2.2H), 3.66 (s, 0.8H), 5.81 (s, 2H), 6.57 (br. s, 4H), 7.13 (m, 2H), 7.22 (m, 1H), 7.35 (m, 1H), 8.67 (m, 1H), 8.87 (dd, 1H).

### Example 3

### Methyl {4,6-diamino-2-[5-fluoro-1-(2-fluorobenzyl)-1H-pyrazolo[3,4-b]pyridin-3-yl]pyrimidin-5-yl}(2,2,2-trifluoroethyl)carbamate

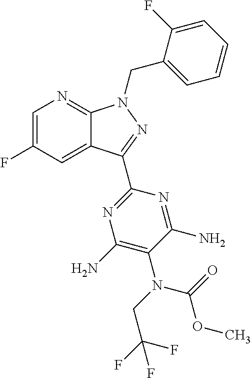 [SMILES: COC(=O)N(CC(F)(F)F)c1c(N)nc(-c2nn(Cc3ccccc3F)c3ncc(F)cc23)nc1N]

A quantity of 3.470 g (8.138 mmol) of the compound from Example 1 was suspended in 35 ml of THF, admixed at 0° C. with 358 mg (8.952 mmol) of sodium hydride (60% suspension in mineral oil) and stirred at 0° C. for 90 min., in the course of which a solution was formed. A quantity of 2.519 g (8.952 mmol) of 2,2,2-trifluoroethyl trichloromethanesulfonate was added and the mixture was stirred at RT for 48 h. It was then stirred with water and concentrated on a rotary evaporator. The residue was taken up in ethyl acetate, and the organic phase was washed twice with water and dried over sodium sulfate. This gave 5.005 g of the target compound (79% of theory, purity by HPLC 65%). A quantity of 250 mg of the residue was purified by means of preparative HPLC (mobile phase: methanol/water, gradient 30:70→90:10).

LC-MS (method 2): Rₜ=0.97 min; MS (EIpos): m/z=509 (M+H)⁺.

¹H-NMR (400 MHz, DMSO-d₆): δ [ppm]=3.63 (s, 3H), 4.06-4.15 (m, 2H), 5.80 (s, 2H), 6.46 (s br, 4H) 7.11-7.15 (m, 2H), 7.20-7.25 (m, 1H), 7.33-7.38 (m, 1H), 8.66 (dd, 1H), 8.91 (dd, 1H).

### B) Assessment of Physiological Efficacy

The suitability of the compounds according to the invention for treating disorders with elevated Hb concentrations can be demonstrated in the following assay systems:

### 1.) In Vitro Assays

1.a.) sGC Enzyme Assay: Stimulation of Recombinant Soluble Guanylate Cyclase (sGC) In Vitro

The investigations on the stimulation of recombinant soluble guanylate cyclase (sGC) by the compounds according to the invention with and without sodium nitroprusside and with and without the heme-dependent sGC inhibitor 1H-1,2,4-oxadiazolo[4,3-a]quinoxalin-1-one (ODQ) are carried out by the method described in detail in the following literature reference: M. Hoenicka, E. M. Becker, H. Apeler, T. Sirichoke, H. Schroeder, R. Gerzer and J.-P. Stasch, “Purified soluble guanylyl cyclase expressed in a baculovirus/Sf9 system: Stimulation by YC-1, nitric oxide, and carbon oxide”, J. Mol. Med. 77 (1999), 14-23. Heme-free guanylate cyclase is obtained by adding Tween 20 to the sample buffer (final concentration 0.5%). Activation of sGC by a test substance is stated as x-fold stimulation of basal activity. Soluble guanylate cyclase (sGC) converts on stimulation GTP into cGMP and pyrophosphate (PPi). PPi is detected with the aid of the assay described below. The signal produced in the assay increases as the reaction progresses and serves as a measure of the sGC enzyme activity under the given stimulation.

To carry out the assay, 29 μl of enzyme solution [0-10 nM soluble guanylate cyclase (prepared according to Hõnicka et al., J. Mol. Med. 77, 14-23 (1999)) in 50 mM TEA, 2 mM MgCl₂, 0.1% BSA (fraction V), 0.005% Brij®, pH 7.5] are initially introduced into a microplate, and 1 μl of the substance to be tested (as a serially diluted solution in DMSO) is added. The mixture is incubated at room temperature for 10 min. Then 20 μl of detection mix [1.2 nM Firefly Luciferase (Photinus pyralis luciferase, Promega), 29 μM dehydroluciferin (prepared according to Bitler & McElroy, Arch. Biochem. Biophys. 72, 358 (1957)), 122 μM luciferin (Promega), 153 μM ATP (Sigma) and 0.4 mM DTT (Sigma) in 50 mM TEA, 2 mM MgCl₂, 0.1% BSA (fraction V), 0.005% Brij®, pH 7.5] are added. The enzyme reaction is started by adding 20 μl of substrate solution [1.25 mM guanosine 5′-triphosphate (Sigma) in 50 mM TEA, 2 mM MgCl₂, 0.1% BSA (fraction V), 0.005% Brij®, pH 7.5] and measured continuously in a luminometer. The extent of the stimulation by the substance to be tested can be determined relative to the signal of the unstimulated reaction.

The activation of heme-free guanylate cyclase is examined by addition of 25 μM of 1H-1,2,4-oxadiazolo[4,3-a]quinoxalin-1-one (ODQ) to the enzyme solution and subsequent incubation for 30 minutes and compared to the stimulation of the native enzyme.

1.b. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

### 1.c Vasorelaxant Effect In Vitro

Rabbits are stunned by a blow to the neck and exsanguinated. The aorta is removed, freed from adhering tissue and divided into rings of a width of 1.5 mm. The rings are placed individually under an initial tension in 5 ml organ baths with Krebs-Henseleit solution which is at 37° C., is gassed with carbogen and has the following composition (in each case mM): sodium chloride 119; potassium chloride: 4.8; calcium chloride dihydrate: 1; magnesium sulfate heptahydrate: 1.4; potassium dihydrogenphosphate: 1.2; sodium bicarbonate: 25; glucose: 10. The force of contraction is detected with Statham UC2 cells, amplified and digitized via A/D converters (DAS-1802 HC, Keithley Instruments, Munich) and recorded in parallel on chart recorders. To produce a contraction, phenylephrine is added to the bath cumulatively in increasing concentration. After several control cycles, the substance to be investigated is added in each further run in increasing dosage in each case, and the height of the contraction is compared with the height of the contraction reached in the last preceding run. The concentration necessary to reduce the height of the control value by 50% is calculated from this (IC₅₀ value). The standard administration volume is 5 μl and the proportion of DMSO in the bath solution corresponds to 0.1%.

### 2.) In Vivo Measurement

### 2.a). Radiotelemetric Measurement of Blood Pressure on Conscious Spontaneously Hypertensive Rats

A commercially available telemetry system from DATA SCIENCES INTERNATIONAL DSI, USA, is employed for the blood pressure measurements on conscious rats described below.

The system consists of 3 main components:
implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The investigations are carried out on adult female spontaneously hypertensive rats (SHR Okamoto) with a body weight of >200 g. SHR/NCrl from the Okamoto Kyoto School of Medicine, 1963 were a cross of male Wistar Kyoto rats with highly elevated blood pressure and female rats having a slightly elevated blood pressure and at F13 handed over to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6.00 am and at 7.00 pm.

### Transmitter Implantation

The telemetry transmitters TA11 PA-C40 used are surgically implanted under aseptic conditions in the experimental animals at least 14 days before the first experimental use. The animals instrumented in this way can be employed repeatedly after the wound has healed and the implant has settled.

For the implantation, the fasted animals are anesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and layered closure of the wound is performed.

An antibiotic (Tardomyocel COMP, Bayer, 1 ml/kg s.c.) is administered postoperatively for prophylaxis of infection.

### Substances and Solutions

Unless indicated otherwise, the substances to be investigated are administered orally by gavage in each case to a group of animals (n=6). The test substances are dissolved in suitable solvent mixtures, or suspended in 0.5% strength Tylose, appropriate for an administration volume of 5 ml/kg of body weight.

A solvent-treated group of animals is employed as control.

### Test Procedure

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch and are switched to transmission in the run-up to the experiment. The emitted signals can be detected online by a data acquisition system (Dataquest™ A.R.T. for Windows, DSI) and be appropriately processed. The data are stored in each case in a file created for this purpose and bearing the experiment number.

In the standard procedure, the following are measured for 10-second periods in each case:
systolic blood pressure (SBP)
diastolic blood pressure (DBP)
mean arterial pressure (MAP)
heart rate (HR)
activity (ACT).

The acquisition of measured values is repeated under computer control at 5-minute intervals. The source data obtained as absolute value are corrected in the diagram with the currently measured barometric pressure (Ambient Pressure Reference Monitor; APR-1) and stored as individual data. Further technical details are given in the extensive documentation from the manufacturing company (DSI).

Unless indicated otherwise, the test substances are administered at 9.00 am on the day of the experiment. Following the administration, the parameters described above are measured over 24 hours.

### Evaluation

After the end of the experiment, the acquired individual data are sorted using the analysis software (Dataquest™ A.R.T.™ Analysis). The blank value is assumed to be the time 2 hours before administration, so that the selected data set includes the period from 7.00 am on the day of the experiment to 9.00 am on the following day.

The data are smoothed over a presettable time by determination of the average (15-minute average) and transferred as a text file to a storage medium. The measured values presorted and compressed in this way are transferred into Excel templates and tabulated. For each day of the experiment, the data obtained are stored in a dedicated file carrying the number of the experiment. Results and test protocols are filed in order in paper form sorted by numbers.

### REFERENCES

Klaus Witte, Kai Hu, Johanna Swiatek, Claudia Müssig, Georg Ertl and Björn Lemme: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994.

### 2.b.) Measurement of Blood Flow and Blood Pressure in Rats

Wistar rats (Hsd Cpb:Wu) of a weight of 250-350 g or ZDF rats (ZDF/Crl-Lepr fa/fa) of a weight of 330-520 g are anesthetized using 2.5% isoflurane in an oxygen/laughing gas mixture (40:60). To determine the blood flow in the carotid artery and the femoral artery, the anesthetized rat is brought into a supine position, and the left carotid artery and the right femoral artery are then carefully exposed. Blood flow is measured by placing flow probes (Transonic Flowprobe) at the vessels. By introducing a PESO artery catheter into the left femoral artery, blood pressure and heart rat are determined (Transducer Ref. 5203660: from Braun CH). The substances are administered as a bole injection or a continuous infusion via a venous catheter in the left femoral vein.

### 2.c.) Assay of Perfusion-Enhancing Substances (Microcirculation)

To reduce perfusion, the right external iliac artery in anesthetized (for example anesthesia by inhalating isoflurane, enflurane) rats (for example ZDF rats) is ligated under sterile conditions. Depending on the degree of collateralization of the animals, it may additionally be necessary to ligate the femoral artery to reduce perfusion. After the operation or else preventatively, the test animals are treated orally, intragastrically (uptake by stomach tube or through feed or drinking water), intraperitoneally, intravenously, intraarterially, intramuscularly, inhalatively or subcutaneously with the test substances. Alternatively, the animals are administered extracellular hemoglobin isolated beforehand from donor animals. The test substances are administered enterally or parenterally, acute or once or more than once per day over a period of up to 5 weeks, or administration is continuous via subcutaneously implanted osmotic mini-pumps (for example Alzet pumps). During the experiment, microperfusion and temperature of the lower extremities are documented. Here, under anesthesia, a temperature-sensitive laser doppler probe (Periflux) is fastened with adhesive to the paws of the rats, allowing the measurement of microperfusion and skin temperature. Depending on the test protocol, samples such as blood (interim diagnostics) and other bodily fluids or organs are removed to carry out further in vitro examinations, or, to document hemodynamics, blood pressure and heart rate are measured via a catheter in the carotid artery. At the end of the experiment, the animals are painlessly sacrificed.

### 2.d.) Assay of Perfusion-Enhancing Substances (Motoric Function) in the Treadmill Test

To determine the motoric function, the running behavior of mice (for example eNOS knock out mice, wild-type mice C-57 B16 or ApoE knock out mice, sickle cell mice, . . . ) is examined on treadmills. To get the mice used to using the treadmill voluntarily, 4-5 weeks before the start of the experiment the animals are put singly into cages with the treadmill and trained. 2 weeks before the start of the experiment, the movements of the mice on the treadmill are recorded by a computer-linked photo cell, and various running parameters such as, for example, daily distance run, individual distances covered, but also their temporal distribution over the day are determined According to their natural running behavior, the animals are randomized into groups (8-12 animals) (control group, sham group and one or more substance groups). Afterwards, the test animals are treated orally, intragastrically (uptake by stomach tube or through feed or drinking water), intraperitoneally, intravenously, intraarterially, intramuscularly, inhalatively or subcutaneously with the test substances. The test substances are administered enterally or parenterally, once or more than once per day over a period of up to 5 weeks, or administration is continuous via subcutaneously implanted osmotic mini-pumps. The running behavior of the animals is monitored and recorded over a period of 5-8 weeks. At the end of the experiment, the animals are painlessly sacrificed. Depending on the test protocol, samples such as blood and other bodily fluids or organs are removed to carry out further in vitro examinations (S. Vogelsberger Neue Tiermodelle für die Indikation Claudicatio Intermittens [Novel animal models for the indication intermittent claudication] (pocket book), publisher: VVB Laufersweiler Verlag (March 2006), ISBN-10: 383595007X, ISBN-13: 978-3835950078).

### 2.e.) Hemodynamics in the Anesthetized Dog

Healthy Mongrel dogs (Marshall BioResources, Marshall Farms Inc; Clyde N.Y.; USA) or Mongrel® dogs suffering from heart failure of both sexes and having a weight of 25-35 kg are used. Anesthesia is initiated by slow i.v. administration of 25 mg/kg sodium thiopental (Trapanal®) and 0.15 mg/kg alcuronium chloride (Alloferin®) and maintained during the experiment by means of a continuous infusion of 0.04 mg/kg*h fentanyl (Fentanyl®), 0.25 mg/kg*h droperidol (Dihydrobenzperidol®) and 15 μg/kg/h alcuronium chloride (Alloferin®). After intubation, the animals are ventilated by the ventilator at a constant respiratory volume such that an end-tidal CO₂ concentration of about 5% is achieved. Ventilation is performed with room air, enriched with about 30% oxygen (normoxia). To measure the hemodynamic parameters, a liquid-filled catheter is implanted into the femoral artery for measuring blood pressure. A Swan-Ganz® catheter having two lumens is introduced in a flow-directed manner via the jugular vein into the pulmonary artery (distal lumen for measuring the pressure in the pulmonary artery, proximal lumen for measuring the central vein pressure). Using a temperature sensor at the tip of the catheter, the continuous cardiac output (CCO) is determined. Blood flow is measured at various vascular beds such as the coronary artery, the carotid artery or the femoral artery by placing flow probes (Transonic Flowprobe) at the vessels in question. The pressure in the left ventricle is measured after introduction of a microtip catheter (Millar® Instruments) via the carotid artery into the left ventricle, and the dP/dt ratio as a measure of contractility is derived therefrom. Substances are administered i.v. via the femoral vein or intraduodenally as cumulative dose/activity curve (bole or continuous infusion). The hemodynamic signals are recorded and evaluated by means of pressure transducers/amplifiers and PONEMAH® as data aquisition software.

To induce heart failure, a pacemaker is implanted into the dogs under sterile conditions. After induction of anesthesia with pentobarbital-Na (15 to 30 mg kg⁻¹ i.v.) followed by intubation and subsequent ventilation (room air; Sulla 808, Dräger®, Germany), anesthesia is maintained by continuous infusion of pentobarbital (1-5 mg kg⁻¹ h⁻¹) and fentanyl (10-40 μg kg⁻¹ h⁻¹). A pacemaker cable (Setrox S60®, Biotronik, Germany) is implanted via an incision of the left jugular vein and placed in the right ventricle. The cable is connected to the pacemaker (Logos®, Biotronik, Germany), which is positioned in a small subcutaneous pocket between the shoulder blades. Ventricular pacing is started only 7 days after the surgical intervention, to obtain heart failure at a frequency of 220 beats/min over a period of 10-28 days.

### 2.f.) Hemodynamics in the Anesthetized Piglet

Hemodynamic parameters were continuously measured by intravascular catheters connected by Combitrans® transducers to Gould® transducers (series 6600) connected to the PoNeMah® acquisition and analysis system.

Healthy Göttingen Minipigs® Ellegaard (Ellegaard, Denmark) of both sexes and having a weight of 2-6 kg were used. Anesthesia was effected by administration of 7.5 mg/ml ketamine, 1.125 mg/ml midazolam (rate of infusion 6-25 ml/h) and 0.6 mg/h pancuronium bromide (Pancuronium®, Organon, Oss, The Netherlands). After intubation, the animals are ventilated by the ventilator at a constant respiratory volume (10-12 ml/kg, 35 breaths/min Avea, Viasys Healthcare, USA) such that an end-tidal CO₂ concentration of about 5% is achieved. Ventilation is performed with room air, enriched with about 40% oxygen (normoxia). For the measurement of the hemodynamic parameters, catheters are inserted into the femoral artery to measure the blood pressure, and a Swan-Ganz® catheter is introduced in a flow-directed manner via the jugular vein into the pulmonary artery (distal lumen for measuring the pressure in the pulmonary artery, proximal lumen for measuring the central vein pressure). Using a temperature sensor at the tip of the catheter, the continuous cardiac output (CCO) is determined. The hemodynamic signals are recorded and evaluated by means of pressure transducers/amplifiers and PONEMAH® as data aquisition software.

### 2.2.) Inhalation of sGC Stimulators and Activators

The experiments were carried out in anesthetized Göttingen minipigs (2.5-4 kg, anesthesia: 7.5 mg/ml of ketamine, 1.125 mg/ml of midazolam (infusion rate 6-25 ml/h) and 0.6 mg/h pancuronium bromid (Pancuronium®, Organon, Oss, The Netherlands)), anesthetized rats and conscious, telemetrically instrumented dogs. After intubation, the anesthetized animals were artificially ventilated with room air enriched with 40% oxygen (pigs: 10-12 ml/kg, 35 breaths/min using Avea, Viasys Healthcare, USA). Acute pulmonary hypertension was induced by an infusion of thromboxane A2 analogs (0.12-0.14 μg/kg/min of 9,11-dideoxy-9α,11α-epoxymethanoprostaglandin F2α (U-46619, Sigma, Germany)) or administration of monocrotaline in rats. The substances were nebulized using the Nebutec® or Aeroneb® Pro nebulizer system or after solid nebulization (Pauluhn) inserted into the inspiration arm of the ventilation. The substances were employed as solids or solutions depending on the molecular structure. The hemodynamic signals are recorded and evaluated by means of pressure transducers/amplifiers and PONEMAH® or CardioMems® as data aquisition software.

### 2.h.) Test of the Pain Threshold/Latency Time in the Hot Plate Test

Hot Plate: Type Socrel DS 37, from Ugo Basile. The measurement is carried out using mice (for example NMRI mice, sickle cell mice). The temperature of the hot plate is 50° C., the maximum duration of the measurement per animal is 90 seconds (cut off time). Immediately prior to the administration of the test substance (test substances for example via feed, drinking water, p.o. administration, Alzet pumps over several days) a hot plate prevalue is determined for each animal Criteria for the latency time (in sec.) to the pain reaction are shaking the paws, licking of the paws or jumping. After about 8 days of test substance administration, the second measurement result is obtained under the same conditions.

1. A method of treatment of sickle cell anemia or for the preservation of blood substitutes, comprising administering a stimulator and/or activator of soluble guanylate cyclase to a human or animal in need thereof.

2. The method of claim 1, wherein the stimulators and/or activators of soluble guanylate cyclase is selected from a compound of formulae (1) to (25) and (26).

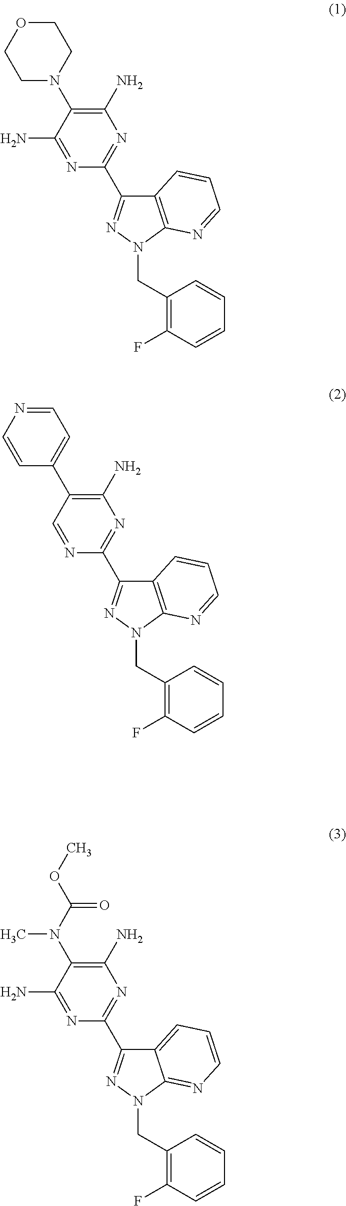 [SMILES: Nc1nc(-c2nn(Cc3ccccc3F)c3ncccc23)nc(N)c1N1CCOCC1 ; Nc1nc(-c2nn(Cc3ccccc3F)c3ncccc23)ncc1-c1ccncc1 ; COC(=O)N(C)c1c(N)nc(-c2nn(Cc3ccccc3F)c3ncccc23)nc1N]

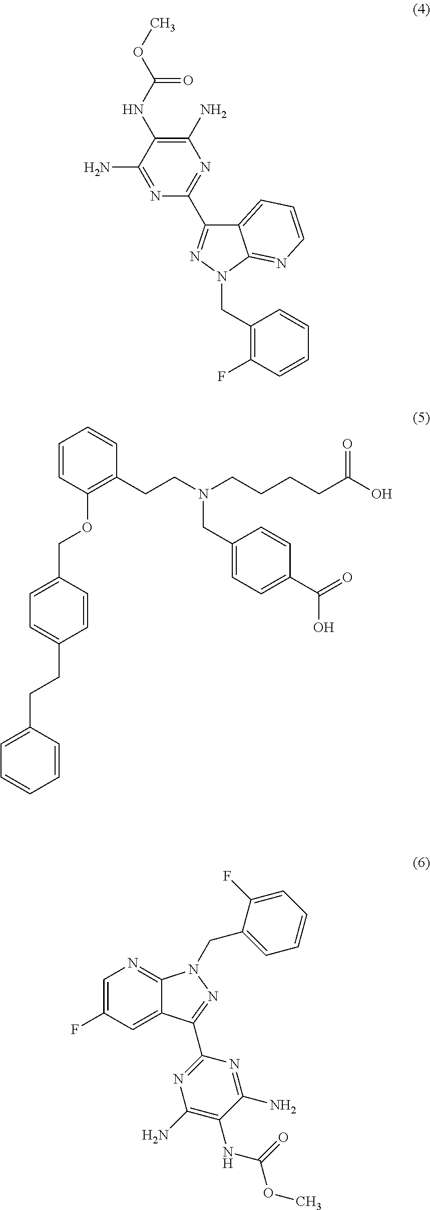 [SMILES: COC(=O)Nc1c(N)nc(-c2nn(Cc3ccccc3F)c3ncccc23)nc1N ; O=C(O)CCCCN(CCc1ccccc1OCc1ccc(CCc2ccccc2)cc1)Cc1ccc(C(=O)O)cc1 ; COC(=O)Nc1c(N)nc(-c2nn(Cc3ccccc3F)c3ncc(F)cc23)nc1N]

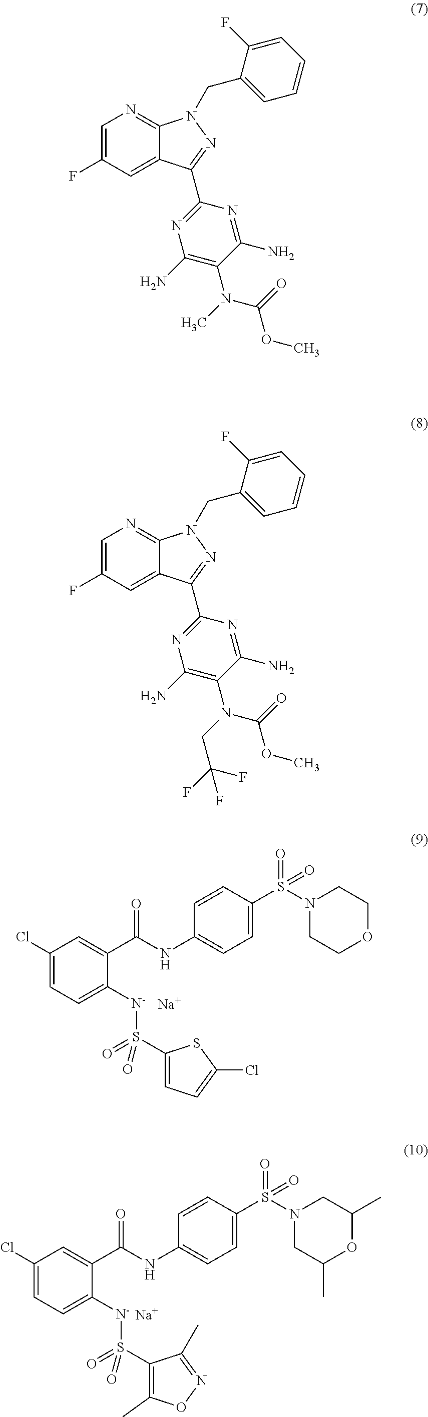 [SMILES: COC(=O)N(C)c1c(N)nc(-c2nn(Cc3ccccc3F)c3ncc(F)cc23)nc1N ; COC(=O)N(CC(F)(F)F)c1c(N)nc(-c2nn(Cc3ccccc3F)c3ncc(F)cc23)nc1N ; O=C(Nc1ccc(S(=O)(=O)N2CCOCC2)cc1)c1cc(Cl)ccc1[N-]S(=O)(=O)c1ccc(Cl)s1 ; [Na+] ; Cc1noc(C)c1S(=O)(=O)[N-]c1ccc(Cl)cc1C(=O)Nc1ccc(S(=O)(=O)N2CC(C)OC(C)C2)cc1 ; [Na+]]

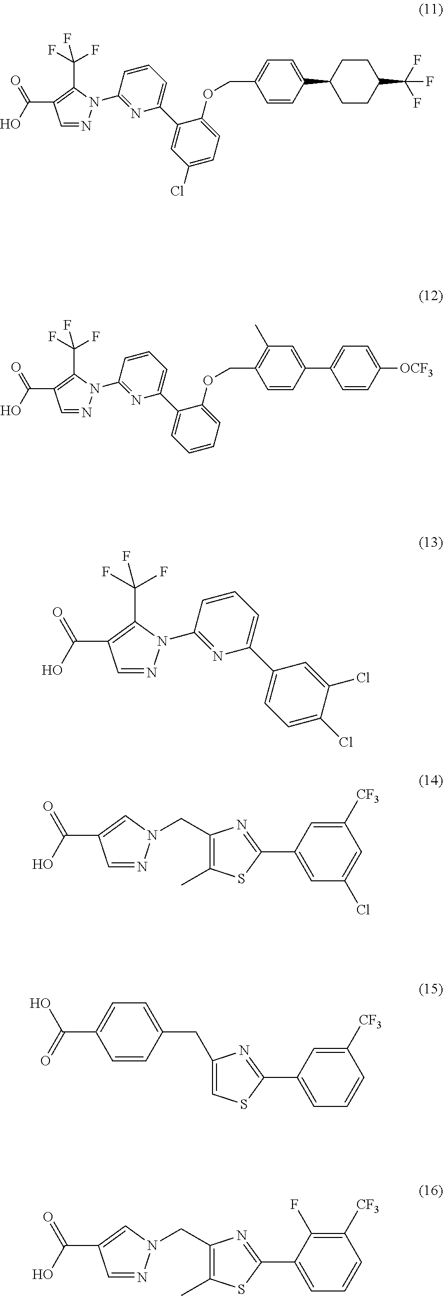

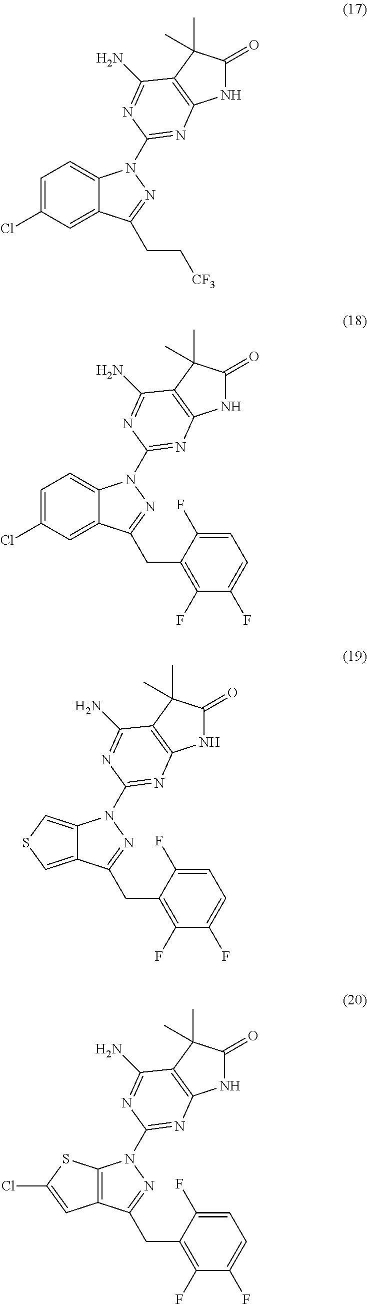

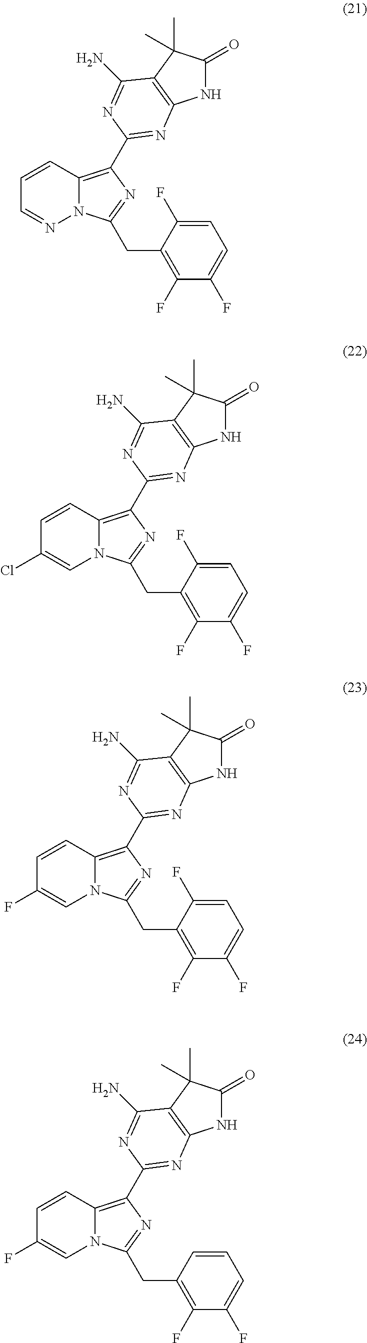 [SMILES: CC1(C)C(=O)Nc2nc(-c3nc(Cc4c(F)ccc(F)c4F)n4ncccc34)nc(N)c21 ; CC1(C)C(=O)Nc2nc(-c3nc(Cc4c(F)ccc(F)c4F)n4cc(Cl)ccc34)nc(N)c21 ; CC1(C)C(=O)Nc2nc(-c3nc(Cc4c(F)ccc(F)c4F)n4cc(F)ccc34)nc(N)c21 ; CC1(C)C(=O)Nc2nc(-c3nc(Cc4cccc(F)c4F)n4cc(F)ccc34)nc(N)c21]

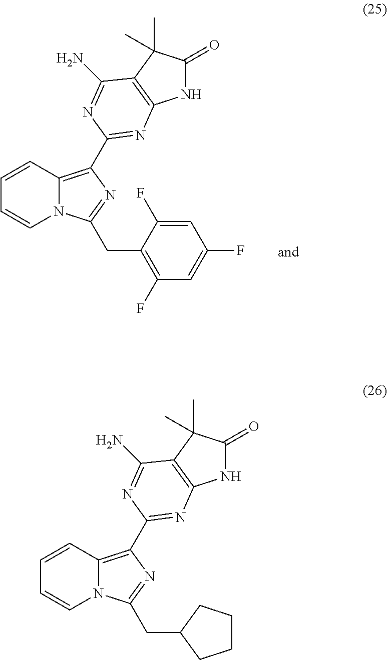 [SMILES: CC1(C)C(=O)Nc2nc(-c3nc(Cc4c(F)cc(F)cc4F)n4ccccc34)nc(N)c21 ; CC1(C)C(=O)Nc2nc(-c3nc(CC4CCCC4)n4ccccc34)nc(N)c21]

3. The method of claim 2, wherein the stimulator and/or activator of soluble guanlyate cyclase is one or more compounds selected from the compound of formulae (3), (4), (5), (6), or (7).

4. (canceled)

5. (canceled)

6. (canceled)

7. (canceled)

8. The method of claim 2, further comprising administering the compounds of formulae (1) to (25) or (26), to the human or animal in combination with at least one PDE5 inhibitor selected from the group consisting of:
tadalafil ((6R,12aR)-2,3,6,7,12,12a-hexahydro-2-methyl-6-(3,4-methylenedioxyphenyl)pyrazino(1′,2′:1,6) pyrido(3,4-b)indole-1,4-dione), vardenafil (2-(2-ethoxy-5-(4-ethylpiperazin-1-yl-1-sulfonyl)phenyl)-5-methyl-7-propyl-3H-imidazo(5,1-f)(1,2,4)triazin-4-one), sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), udenafil 5-[2-propyloxy-5-(1-methyl-2-pyrrolidinylethylamidosulfonyl)phenyl]-methyl-3-propyl-1,6-dihydro-7H-pyrazolo(4,3-d)pyrimidin-7-one, dasantafil (7-(3-bromo-4-methoxybenzyl)-1-ethyl-8-[[(1,2)-2-hydroxycyclopentyl]amino]-3-(2-hydroxyethyl)-3,7-dihydro-1-purine-2,6-dione), avanafil (4-{[(3-chloro-4-methoxyphenyl)methyl]amino}-2-[(2S)-2-(hydroxymethyl)pyrrolidin-1-yl]-N-(pyrimidin-2-ylmethyl)pyrimidine-5-carboxamide), mirodenafil, lodenafil, UK 369.003, UK 371.800, SLx 2101 of Surface Logix, and LAS 34179 (triazolo[1,2-]xanthine, 6-methyl-4-propyl-2-[2-propoxy-5-(4-methylpiperazino)sulfonyl]phenyl) or salts, hydrates or hydrates of the salts thereof.

9. The method of claim 2, wherein a compound of formulae (3), (4), (5), (6), or (7) is administered to the human or animal in combination with vardenafil and/or sildenafil.

10. (canceled)

11. (canceled)

12. (canceled)

13. (canceled)

14. The method of claim 1, wherein the stimulator and/or activator of soluble guanylate cyclase is administered by inhalation.

15. (canceled)
